(19) United States
(12) Patent Application Publication
(10) Pub. No.: US 2026/0216233 A1
(43) Pub. Date: Jul. 30, 2026

(54) COMPOSITIONS FOR THE PREVENTION AND/OR TREATMENT OF COLONIC GROUP 3 INNATE LYMPHOID CELLS (ILC3)-MEDIATED DISEASES AND METHODS OF USING SAME

(71) Applicant: Duke University, Durham, NC (US)

(72) Inventors: Neeraj Surana, Durham, NC (US); Chin Yee Tan, Durham, NC (US)

(21) Appl. No.: 19/147,598

(22) PCT Filed: Jan. 17, 2024
(86) PCT No.: PCT/US2024/011808

§ 371 Date: Jul. 11, 2025

Related U.S. Application Data
(60) Provisional application No. 63/439,394, filed on Jan. 17, 2023.

(51) Int. Cl. A61K 31/736; A61K 35/00; A61K 35/742; A61K 45/06; A61P 1/04; A61P 31/04; C08B 37/00

(52) U.S. Cl. CPC A61K 31/736; A61K 35/742; A61K 45/06; A61P 1/04; A61P 31/04; C08B 37/0087; A61K 2035/115

(57) ABSTRACT

The present disclosure describes, in part, compositions for the prevention and/or treatment of group 3 innate lymphoid cells (ILC3)-mediated diseases and methods of using same.

### II. CROSS-REFERENCE TO RELATED APPLICATIONS

This application claims the benefit of U.S. Provisional Application No. 63/439,394 filed 17 Jan. 2023, which is incorporated by reference herein in its entirety.

### I. STATEMENT REGARDING FEDERALLY SPONSORED RESEARCH

This invention was made with Government support under Federal Grant No. R03AI142341 awarded by the National Institutes of Health. The Federal Government has certain rights to this invention.

### III. REFERENCE TO THE SEQUENCE LISTING

The Sequence Listing submitted 17 Jan. 2024 as a XML file named “23_3004_WO_Sequence_Listing”, created on 17 Jan. 2024 and having a size of 30 kilobytes is hereby incorporated by reference pursuant to 37 C.F.R. § 1.52(e)(5).

### IV. BACKGROUND OF THE INVENTION

The gut microbiota plays a fundamental role in controlling many facets of human physiology, with impacts on the immune system being chief among them. Although there have been increasing successes in identifying disease-regulating commensal bacteria, the mechanisms by which they work remain poorly understood.

Accordingly, there is an urgent and previously unmet need to identify disease-regulating commensal bacteria and decipher the mechanism by which they impact the immune system.

### V. BRIEF SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Previously, the inventors identified Clostridium immunis, a new human-derived commensal bacterial species that modulates colitis. The present disclosure is based, in part, on the characterization of the immunological factors and specific bacterial determinant required for its disease modulating activity. As shown herein, C. immunis directly regulates the effector function of group 3 innate lymphoid cells (ILC3s), with altered levels of IL-22 and GM-CSF in states of health and inflammation, respectively. This impact on ILC3s serves as a common immunological node for modulation of visceral adiposity and intestinal inflammation. Surprisingly, the two bacterial species most closely related to C. immunis lack this ILC3-modulating activity. In another embodiment, and guided by comparative genomics, the present disclosure further provides for a purified exopolysaccharide (EPS) that recapitulates the immunomodulatory activity of C. immunis in vitro and in vivo. Structural analysis revealed that phosphocholine on the EPS correlated with activity, and deletion of the C. immunis phosphocholine biosynthesis locus (LicABC) resulted in functionally inactive EPS. The results reveal that a phosphocholine moiety present on a bacterial polysaccharide is critically required to orchestrate ILC3 effector function and regulate intestinal homeostasis.

Accordingly, one aspect of the present disclosure provides a purified exopolysaccharide (EPS) isolated from C. immunis, said EPS comprising, consisting or, or consisting essentially of a plurality of mannose and glucose subunits and a phosphocholine.

In an embodiment, the phosphocholine is present at 3.22 ppm on a 1H NMR spectra.

In an aspect, the isolated EPS can comprise peaks at 160 kDa, 88 kDa, 35 kDa, 26 kDa, and 5 kDa when analyzed for distribution size through size exclusion chromatography (SEC). In an aspect, the isolated EPS has a mannan-like structure. In an aspect, the backbone of the purified EPS predominantly can comprise mannose.

In an aspect the purified EPS's spectroscopy (COSY) spectra can show a cross peak between the phosphocholine protons at 4.33 and 3.65 ppm.

Another aspect of the present disclosure provides a pharmaceutical composition comprising a purified EPS as provided herein and a pharmaceutically acceptable diluent, excipient and/or carrier.

Another aspect of the present disclosure provides a method of reducing the amount of, activity of, and/or proliferation of colonic group 3 innate lymphoid cells (ILC3s) in a subject, the method comprising, consisting of, or consisting essentially of administering to the subject a therapeutically effective amount of a purified EPS as provided herein, or a pharmaceutical composition thereof, such that the amount of, activity of, and/or proliferation of ILC3 cells are reduced in the subject. In an embodiment, a disclosed method can further comprise administering to the subject one or more additional therapeutic agents. In another aspect, a disclosed method can further comprise co-administering to the subject one or more additional therapeutic agents prior to, concurrently with, or after the administration of the purified EPS or a pharmaceutical composition thereof. In an embodiment, the disclosed one or more additional therapeutic agents can be administered prior to the purified EPS or pharmaceutical composition thereof. In another embodiment, the disclosed one or more additional therapeutic agents can be administered concurrently with the purified EPS or pharmaceutical composition thereof. In another embodiment, the disclosed one or more additional therapeutic agents can be administered after the purified EPS or pharmaceutical composition thereof.

Another aspect of the present disclosure provides a method of treating or preventing an ILC3-mediated disease in a subject, the method comprising, consisting of, or consisting essentially of administering to the subject a therapeutically effective amount of a purified EPS as provided herein, or a pharmaceutical composition thereof, such that the ILC3-mediated disease is treated and/or prevented in the subject. In an embodiment, the method can further comprise administering to the subject one or more additional therapeutic agents. In another aspect the method can further comprise co-administering to the subject one or more additional therapeutic agents prior to, concurrently with, or after the administration of the purified EPS or a pharmaceutical composition thereof. In an embodiment, the one or more additional therapeutic agents is administered prior to the purified EPS or pharmaceutical composition thereof. In another embodiment, the one or more additional therapeutic agents is administered concurrently with the purified EPS or pharmaceutical composition thereof. In an embodiment, the one or more additional therapeutic agents is administered after the purified EPS or pharmaceutical composition thereof.

In an embodiment, the ILC3-mediated disease can be inflammatory bowel disease, psoriasis, asthma, allergy-induced asthma, obesity, multiple sclerosis, enteric infections, necrotizing enterocolitis, and combinations thereof. In an embodiment, the ILC3-mediated disease is selected from the group consisting of inflammatory bowel disease, psoriasis, asthma, allergy-induced asthma, obesity, multiple sclerosis, enteric infections, necrotizing enterocolitis, and combinations thereof.

In an embodiment, the ILC3-mediated disease can comprise obesity.

In an embodiment, the ILC3-mediated disease can comprise IBD. In an embodiment, the IBD is selected from the group consisting of Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease, Behcet's disease and combinations thereof.

In another embodiment, the method can further comprise administering to the subject a pre-biotic. In an embodiment, the prebiotic is administered prior to the purified EPS or pharmaceutical composition thereof. In another embodiment, the prebiotic is administered concurrently with the purified EPS or pharmaceutical composition thereof. In an embodiment, the prebiotic is administered after the purified EPS or pharmaceutical composition thereof.

Another aspect of the present disclosure provides a method of treating or preventing metabolic syndrome (MetS) in a subject, the method comprising, consisting of, or consisting essentially of administering to the subject a therapeutically effective amount of a purified EPS as provided herein, or a pharmaceutical composition thereof, such that the metabolic syndrome (MetS) is treated and/or prevented in the subject. In an embodiment, the method further can comprise administering to the subject one or more additional therapeutic agents. In another aspect, the method can further comprise co-administering to the subject one or more additional therapeutic agents prior to, concurrently with, or after the administration of the purified EPS or a pharmaceutical composition thereof. In an embodiment, the one or more additional therapeutic agents is administered prior to the purified EPS or pharmaceutical composition thereof. In another embodiment, the one or more additional therapeutic agents is administered concurrently with the purified EPS or pharmaceutical composition thereof. In an embodiment, the one or more additional therapeutic agents is administered after the purified EPS or pharmaceutical composition thereof.

In an aspect, the MetS can comprise one or more conditions comprising increased waist circumference, high blood pressure, elevated serum triglyceride and glucose levels, decreased high-density lipoprotein (HDL) levels, and any combination thereof. In an aspect, the MetS can comprise conditions selected from the group consisting of increased waist circumference, high blood pressure, elevated serum triglyceride and glucose levels, decreased high-density lipoprotein (HDL) levels, and combinations thereof.

Another aspect of the present disclosure provides a method of decreasing visceral adiposity in a subject, the method comprising, consisting of, or consisting essentially of administering to the subject a therapeutically effective amount of a purified EPS, or a pharmaceutical composition thereof, such that the visceral adiposity is decreased in the subject.

Another aspect of the present disclosure provides a method of decreasing visceral fat in a subject, the method comprising, consisting of, or consisting essentially of administering to the subject a therapeutically effective amount of a purified EPS, or a pharmaceutical composition thereof, such that the visceral fat is decreased in the subject.

Another aspect of the present disclosure provides a method of inhibiting and/or decreasing the expression of a gene selected from the group consisting of Cd36, Scd1, Nfil3 and any target genes thereof, in a cell, the method comprising, consisting of, or consisting essentially of contacting the cell with an effective amount of a purified EPS as provided herein such that the expression of the gene and/or any target genes thereof are decreased and/or inhibited in the cell.

Another aspect of the present disclosure provides a method of inhibiting and/or decreasing the expression of a gene selected from the group consisting of Cd36, Scd1, Nfil3 and any target genes thereof, in a subject, the method comprising, consisting of, or consisting essentially of administering to the subject a therapeutically effective amount of a purified EPS as provided herein, or a pharmaceutical composition thereof, such that the expression of the gene and/or any target genes thereof are decreased and/or inhibited in the subject.

Another aspect of the present disclosure provides a method of increasing the expression of the gene Csf2 and any target genes thereof, in a cell, the method comprising, consisting of, or consisting essentially of contacting the cell with an effective amount of a purified EPS as provided herein such that the expression of the Csf2 gene and/or any target genes thereof are increased in the cell.

Another aspect of the present disclosure provides a method of increasing the expression of the gene Csf2, and any target genes thereof, in a subject, the method comprising, consisting of, or consisting essentially of administering to the subject a therapeutically effective amount of a purified EPS as provided herein, or a pharmaceutical composition thereof such that the expression of the Csf2 gene and/or any target genes thereof are increased in the subject.

In an embodiment of the invention, a disclosed kit is provided comprising the purified EPS, or a pharmaceutical composition thereof. In an embodiment, a disclosed kit can comprise the purified EPS, or a pharmaceutical composition thereof, and one or more components. In one embodiment, the one or more components are provided in combinations. In an embodiment, a disclosed kit can further comprise a suitable container. In an embodiment, the one or more components can comprise reagents, selected from the group comprising dilution buffers, reconstitution solutions, wash buffers, storage buffers, and control reagents. In an embodiment, a disclosed kit can further comprise instructions for using the components. In an embodiment, the instructions can be recorded on a suitable recording medium and are present in the kit as a package insert, in the labeling of the container of the kit or components thereof. In an embodiment, the instructions can be present as an electronic storage data file present on a suitable computer readable storage medium.

In an embodiment, a disclosed kit can comprise one or more therapeutic agents. In an embodiment, the disclosed one or more therapeutic agents can comprise at least one of a biological active agent, a pharmaceutical active agent, an anti-bacterial agent, an anti-fungal agent, an anti-viral agent, a corticosteroid, an analgesic, or a combination thereof. In an embodiment, a disclosed kit can comprises one or more prebiotics.

In an embodiment, the purified exopolysaccharide (EPS) from C. immunis can be isolated in accordance with the following steps: culturing medium inoculated with C. immunis; centrifuging the cultured medium to pellet the C. immunis; filtering the supernatant; treating the filtered supernatant with DNaseI and RNaseA; centrifuging the DNaseI and RNaseA treated supernatant to clarify; treating the clarified supernatant with proteinase; dialyzing the proteinase treated supernatant by adding ultrapure water and spinning through a spin column to eliminate the proteinase; and precipitating the proteinase-free and dialyzed remaining solution with ethanol.

In an aspect, the culturing medium inoculated with C. immunis can be carried out for between about 2 hrs. and about 30 hrs., more preferably between about 5 hrs. and about 20 hrs., and most preferably between about 8 hrs. and about 15 hrs.

In an aspect, the filtering of the supernatant can be carried out through a 0.45 μm filter, and then through a 0.22 μm filter.

In an aspect, the concentrating the supernatant can be carried out multiple times using a 100 kDa molecular weight cutoff spin column.

In an aspect, the treating the filtered supernatant with DNaseI and RNaseA can be carried out for between about 2 hrs. and about 30 hrs., more preferably between about 5 hrs. and about 20 hrs., and most preferably between about 8 hrs. and about 15 hrs.

In an aspect, the treating the clarified supernatant with proteinase can be carried out with proteinase K for between about 2 hrs. and about 30 hrs., more preferably between about 5 hrs. and about 20 hrs., and most preferably for between about 8 hrs. and about 15 hrs.

In an aspect, the dialyzing the proteinase treated supernatant by adding ultrapure water and spinning through a spin column to eliminate the proteinase can be carried out with a 100 kDa cutoff spin column.

In an aspect the adding ultrapure water and spinning through a spin column can be carried out multiple times.

In an aspect, the precipitating the proteinase-free and dialyzed remaining solution with ethanol can be carried out for between about 2 hrs. and about 30 hrs., more preferably for between about 5 hrs. and about 20 hrs., and most preferably for between about 8 hrs. and about 15 hrs.

In an aspect, a disclosed method can further comprise the step of quantifying the yield of purified EPS.

In an aspect, disclosed herein is a method of treating or preventing cancer in a subject, the method comprising: administering to the subject a therapeutically effective amount of the disclosed purified EPS, or the pharmaceutical composition, such that the cancer is treated and/or prevented in the subject. In an aspect, the method can further comprise co-administering to the subject one or more additional therapeutic agents. In an aspect, the one or more additional therapeutic agent can be an immunotherapy agent.

Another aspect of the present disclosure provides all that is described and illustrated herein.

### VI. BRIEF DESCRIPTION OF THE FIGURES

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1 is data showing C. immunis modulates disease via ILC3s in accordance with an embodiment of the present disclosure. FIG. 1A, Volcano plot depicting differentially expressed genes in colons from MMb mice treated orally with or without C. immunis (n=3 mice per group). Genes highlighted in red are related to lipid uptake and metabolism. FIG. 1B, Gonadal fat mass normalized to body weight from MMb mice treated orally with or without the indicated bacteria (n=4-6 mice per group). FIGS. 1C-FIG. 1E, Quantitative PCR (qPCR) analysis of small-intestinal Cd36 expression (FIG. 1C; n=3-8 mice per group), gonadal fat mass normalized to body weight (FIG. 1D; n=5-10 mice per group), and body weight (FIG. 1E; n=10-14 mice per group) from ILC3-sufficient (Cre⁻) or ILC3-deficient (Cre⁺) mice treated orally with or without C. immunis. FIG. 1F-FIG. 1H, Weight change (FIG. 1F; n=8-9 mice per group), colon length (FIG. 1G; n=4-7 mice per group), and histological assessment of disease severity in the colon at day 7 (FIG. 1H; n=3-5 mice per group) from ILC3-sufficient or ILC3-deficient mice treated orally with or without C. immunis and subjected to DSS-induced colitis. Data are pooled from two (FIG. 1B, FIG. 1C, FIG. 1D, FIG. 1F) or three (FIG. 1E) experiments or are representative of ≥2 independent experiments (FIG. 1G, H). Data are represented as mean±s.e.m. *P<0.05; **P<0.01; ****P<0.0001; ns, not significant by Brown-Forsyth and Welch ANOVA with Dunnett's T3 multiple comparisons test (FIG. 1B, FIG. 1D), one-way ANOVA with Holm-Šidák correction (FIG. 1C, FIG. 1E), comparison of fit by non-linear regression (FIG. 1F), or a Kruskal-Wallis test with correction for multiple comparisons (FIG. 1G, H).

FIG. 2 is data showing C. immunis contextually regulates ILC3 effector function in accordance with an embodiment of the present disclosure. FIG. 2A, qPCR analysis of Csf2 in MNK-3 cells incubated with a control (sterile culture media) or C. immunis supernatant. FIG. 2B, FIG. 2C, qPCR analysis of small-intestinal expression of Csf2 (FIG. 2B) and Il22 (FIG. 2C) in mice orally treated with or without C. immunis (n=6-7 mice per group). FIG. 2D, FIG. 2E, qPCR analysis of colonic expression of Csf2 (FIG. 2D) and Il22 (FIG. 2E) in mice orally treated with or without C. immunis and challenged with DSS-induced colitis for 7 days (n=5-8 mice per group). FIG. 2F, FIG. 2G, Gonadal fat mass normalized to body weight in Csf2rb^{-/-} mice (f; n=11 mice per group) or Rag1^{-/-} mice treated with an IL-22 depleting antibody (FIG. 2G; n=8 mice per group). Mice were orally treated with or without C. immunis. H-j, Weight change (FIG. 2H, FIG. 2J) and colon length (FIG. 21, FIG. 2K) of Csf2rb^{-/-} mice (FIG. 2H, FIG. 2I; n=10 mice per group) or mice treated with an IL-22 depleting antibody (FIG. 2J, FIG. 2K; n=6 mice per group). Mice were treated with or without C. immunis and subjected to DSS-induced colitis. Data are pooled from two (FIG. 2F, FIG. 2H, FIG. 2I) or three (FIG. 2A) experiments or are representative of ≥2 experiments (FIG. 2B, FIG. 2C, FIG. 2D, FIG. 2E, FIG. 2G, FIG. 2J, FIG. 2K). Data are represented as mean±s.e.m. *P<0.05; **P<0.01; ns, not significant by Mann Whitney U test (FIG. 2A-FIG. 2E, FIG. 2I, FIG. 2K), unpaired t-test (FIG. 2F, FIG. 2G), or comparison of fit by non-linear regression (FIG. 2H, FIG. 2J).

FIG. 3 is data showing a purified exopolysaccharide recapitulates the activity of C. immunis in accordance with an embodiment of the present disclosure. FIG. 3A, qPCR analysis of Csf2 expression in MNK-3 cells incubated with a control (sterile culture media) or supernatants from the indicated bacteria. FIG. 3B, Schematic of comparative genomics analysis for C. immunis genes associated with activity. FIG. 3C, Periodic acid-Schiff-stained SDS-PAGE gel of a control, C. immunis supernatant, and purified C. immunis exopolysaccharide (EPS). The control included sterile bacterial media that went through the same purification process as C. immunis EPS, and all samples represent an equal amount of culture volume. FIG. 3D, qPCR analysis of Csf2 expression in MNK-3 cells incubated with or without C. immunis EPS. FIG. 3E-FIG. 3G, qPCR analysis of small-intestinal expression of Nfil3 (FIG. 3E), Cd36 (FIG. 3F), and Scd1 (FIG. 3G) in Rag1^{-/-} mice treated with control or EPS isolated from C. immunis or C. symbiosum (n=5-9 per group). FIG. 3H, Gonadal fat mass normalized to body weight in Rag1^{-/-} mice treated with control or EPS isolated from C. immunis or C. symbiosum (n=10-17 per group). FIG. 3I, Survival of Swiss Webster MMb mice treated with a control (sterile culture media), C. immunis, or C. immunis EPS following DSS-induced colitis (n=5-19 per group). Data are pooled from two (FIG. 3D-H) or three experiments (FIG. 3I) or are representative of 2 (FIG. 3A) or 3 experiments (FIG. 3C). Data are represented as mean±s.e.m. *P<0.05; **P<0.01; ***P<0.001; ns, not significant by Kruskal-Wallis test with Dunn's multiple comparisons test (FIG. 3A, E-H), unpaired t-test (FIG. 3D), or log-rank test (FIG. 3I).

FIG. 4 is data showing the addition of phosphocholine to C. immunis EPS is critical for its function in accordance with an embodiment of the present disclosure. FIG. 4A, ¹H-NMR spectra of EPS purified from C. immunis, C. symbiosum, or C. immunis ΔLicABC. The arrow highlights a peak corresponding to phosphocholine, which is only present in EPS from C. immunis. FIG. 4B, Immunoblot of EPS purified from C. immunis (CiEPS) or C. immunis ΔLicABC (CiΔLicABCEPS) that is probed with an antibody against phosphocholine (ChoP; top blot). Samples were also visualized with periodic acid-Schiff stain (PAS; bottom blot). FIG. 4C, Quantification of the band intensities in panel FIG. 4B, with values from the immunoblot normalized to that from the PAS-stained blot. FIG. 4D, qPCR analysis of Csf2 expression in MNK-3 cells incubated with a control (sterile culture media) or supernatants from C. immunis or C. immunis ΔLicABC. FIG. 4E-FIG. 4G, qPCR analysis of small-intestinal Nfil3 (FIG. 4E), Cd36 (FIG. 4F), and Scd1 (FIG. 4G) in Rag1^{-/-} mice treated with a control or CiΔLicABCEPS. FIG. 4H, Gonadal fat mass normalized to body weight in Rag1^{-/-} mice treated with control or CiΔLicABCEPS (n=6-7 mice per group). Data are pooled from 3 experiments (FIG. 4C, D) or are representative of ≥2 experiments (FIG. 4B, E-H). Data are represented as mean±s.e.m. *P<0.05; **P<0.01; ns, not significant by unpaired t-test (FIG. 4C), Kruskal-Wallis test with Dunn's multiple comparisons test (FIG. 4D) or Mann Whitney U test (FIG. 4E-H).

FIG. 5 is data showing that C. immunis protects against NEC in accordance with an embodiment of the present disclosure. FIG. 5A, FIG. 5B. 7-day-old pups were subjected to the hypoxia-gavage model of NEC, where the addition of pathogenic bacteria (“NECteria”) is required to induce disease. Pups were treated with or without C. immunis, and weights (FIG. 5A) and small-intestinal histology at day 3 (FIG. 5B) are presented. FIG. 5C. 1-day-old pups were treated with sterile media (control) or C. immunis and challenged with DSS-induced NEC. Survival is shown.

FIG. 6 is data showing that C. immunis decreases obesity in accordance with an embodiment of the present disclosure. FIG. 6A, FIG. 6B. Mice that are deficient in ILC3s (Ahrfl/fl Rorc-Cre) and their littermate controls were orally treated with or without C. immunis. Body weight (FIG. 6A) and visceral fat (FIG. 6B) are shown. FIG. 6C. MMb mice were orally treated with C. immunis, C. symbiosum, or C. clostridioforme, and gonadal fat was assessed 1 week later.

FIG. 7 is data showing that purified C. immunis exopolysaccharide is biologically active in accordance with an embodiment of the present disclosure. Mice were orally treated with sterile media (control), C. immunis, or C. immunis polysaccharides isolated from the supernatant. FIG. 7A. Gonadal fat was assessed 1 week later. FIG. 7B. Mice were challenged with DSS colitis, in which C. immunis protects against mortality. Survival is shown.

FIG. 8 is a graph showing that C. immunis inhibits expression of small-intestinal lipid metabolism genes in accordance with an embodiment of the present disclosure. qPCR analysis of small-intestinal expression of Scd1 (FIG. 8A) and Cd36 (FIG. 8B) in MMb mice orally treated with or without C. immunis (n=4-7 mice per group). Data are pooled from two experiments and represent mean±s.e.m. *P<0.05 by unpaired t test.

FIG. 9 are graphs showing C. immunis decreases expression of small-intestinal Nfil3 and its target genes in accordance with an embodiment of the present disclosure. FIG. 9A, qPCR analysis of small-intestinal Nfil3 expression in MMb mice orally treated with the indicated bacteria (n=4-6 mice per group). Data are pooled from two experiments and represent mean±s.e.m. FIG. 9B, Gene set enrichment analysis for NFIL3-dependent genes. NES, normalized enrichment score. **P<0.01; ns, not significant by ANOVA with Holm-Šidák correction for multiple comparisons.

FIG. 10 is a graph showing that C. immunis decreases adiposity independent of adaptive immunity in accordance with an embodiment of the present disclosure. Gonadal fat mass normalized to body weight of Rag1^{-/-} mice orally treated with or without C. immunis (n=4-5 mice per group). Data is representative of two experiments and represent mean±s.e.m. **P<0.01 by unpaired t test.

FIG. 11 is data showing that C. immunis does not alter expression of Il1b or Il23 in accordance with an embodiment of the present disclosure. qPCR analysis of Il1b (FIG. 11A) and Il23 (FIG. 11B) expression in colonic tissue obtained from MMb mice orally treated with or without C. immunis (n=6 mice per group). Data are pooled from two independent experiments and represent mean±s.e.m. ns, not significant by Mann Whitney U test.

FIG. 12 are graphs showing that C. immunis affects expression of select cytokine genes in MNK-3 cells in accordance with an embodiment of the present disclosure. qPCR analysis of Il22 (FIG. 12A) and Il17a (FIG. 12B) in MNK-3 cells incubated with a control (sterile culture media) or C. immunis supernatant. Data are pooled from two experiments and represent mean±s.e.m. **P<0.01; ns, not significant by Mann Whitney U test.

FIG. 13 is data showing that C. immunis supernatant protects MMb mice from colitis-associated death in accordance with an embodiment of the present disclosure. Survival of Swiss Webster MMb mice orally treated with a control (sterile culture media) or C. immunis supernatant (n=10-15 per group) and subjected to DSS-induced colitis. Data are pooled from three independent experiments. *P<0.05 by log-rank test.

FIG. 14 is data showing that purified C. immunis EPS is devoid of significant protein contamination in accordance with an embodiment of the present disclosure. Silverstained SDS-PAGE gel of a control, C. immunis supernatant, and C. immunis EPS. The control included sterile bacterial media that went through the same purification process as C. immunis EPS, and all samples represent an equal amount of culture volume.

FIG. 15 is data showing that EPS isolated from C. immunis, C. symbiosum and C. immunis ΔLicABC have comparable monosaccharide composition and ¹H-NMR signals from the carbohydrate portion in accordance with an embodiment of the present disclosure. FIG. 15A, Glycosyl composition analysis of EPS isolated from C. immunis, C. symbiosum and C. immunis ΔLicABC. FIG. 15B-FIG. 15D, ¹H-NMR spectra of EPS samples from C. immunis (FIG. 15B), C. symbiosum (FIG. 15C) or C. immunis ΔLicABC (FIG. 15D). The inset shows a zoomed in view of the anomeric signals that form the carbohydrate portion.

FIG. 16 is data showing that C. immunis capsular polysaccharide operon organization and deletion strategy in accordance with an embodiment of the present disclosure. FIG. 16A, A schematic of the C. immunis genetic locus containing LicABC and flanking genes. The predicted promoter is indicated by the arrow. FIG. 16B, A schematic of the genetic strategy used to delete LicABC in C. immunis. E. coli was used to transfer a suicide plasmid containing an erythromycin resistance gene (ErmB) flanked by regions homologous to LicA into C. immunis via conjugation. Erythromycin-resistant (EryR) C. immunis mutants contained the ErmB gene and an 80-bp deletion in LicA.

FIG. 17 is data showing LicABC genes are present in taxonomically diverse commensal organisms in accordance with an embodiment of the present disclosure. LicABC genes homologous to the C. immunis locus were identified by BLAST and are depicted in a randomized accelerated maximum likelihood (RAxML) phylogenetic tree, with the LicABC genes from four bacteria primarily localized to the respiratory tract added in for reference.

FIG. 18 shows size exclusion chromatography (SEC) of EPS isolated from C. immunis, C. immunis ΔlicABC, and C. symbiosum demonstrating that the C. immunis EPS contains polysaccharides of different sizes.

FIG. 19 shows linkage analysis demonstrating 4 major types of monosaccharides present in a roughly 1:1:1:1 ratio (Man, mannose; Glc, glucose; Rha, rhamnose; Gal, galactose; GalA, galacturonic acid. Numbers in panel B refer to linkages).

FIG. 20 shows the COSY spectra of the C. immunis EPS compared to that of C. immunis_{ΔlicABC}. Note that C. immunis EPS shows a cross peak between the phosphocholine protons at 4.33 ppm and 3.65 ppm, which were absent in the C. immunis_{ΔlicABC}.

FIG. 21 presents heteronuclear single quantum coherence (HSQC) experimental results showing the anomeric regions in the HSQC spectra of C. immunis and C. immunis_{ΔliABC} EPS. The anomeric CH are labelled with letters in the C. immunis EPS spectra.

FIG. 22 presents zoomed anomeric region in the ¹H NMR spectra of C. immunis and C. immunis_{ΔlicABC} EPS, with tentative assignments of the anomeric protons (as labeled in FIG. 21 above). Superscript a and b denote residues within a linear or branched region, respectively.

FIG. 23 presents a tumor growth graph showing that co-administration of C. immunis as Probiotics to mice subcutaneously implanted with MC38 cells enhances the efficacy of anti-PD1 cancer therapy.

FIG. 24A presents a weight change curve demonstrating that C. immunis protects against necrotizing enterocolitis. Rag+/− pups (~7 days old) were treated with hypoxia and intestinal pathogens (NECteria) as per Gopalakrishna et al. (Gopalakrishna, K. P., et al. (2019) Nat. Med. 25:1110-1115) to induce necrotizing enterocolitis. Mice were treated with either a control or C. immunis and body weight was monitored over 3 days. The C. immunis treated pups had body weight gain similar to control animals (that did not receive the NECteria organisms) and, as shown in FIG. 24B, lower inflammation on intestinal histology compared to the control animals.

FIG. 25A, FIG. 25B, and FIG. 25C present survival curves demonstrating that C. immunis protects against a second model of necrotizing enterocolitis. Neonatal mice were administered dextran sodium sulfate (DSS) by oral gavage, which causes them to die (FIG. 25A), consistent with earlier studies demonstrating that DSS results in a NEC-like phenotype in neonatal pups (Ginzel M, et al. (2017) PLoS One. 12(8)). Pups treated with C. immunis were protected against disease (FIG. 25B), and this protection required the organism to be viable (FIG. 25C). Of note, these data indicate that any polysaccharide that is present on the organism is functionally inactive, otherwise the heat-killed organism should have retained activity.

FIG. 26 present a weight change curve demonstrating that C. immunis protects against enteric infection with Clostridioides difficile. Mice were treated with or without C. immunis starting 1-week prior to infection. At day 0, all mice were orally infected with C. difficile, and weights were monitored for 5 days. Mice treated with C. immunis had less weight loss than control mice.

### VII. DETAILED DESCRIPTION OF THE INVENTION

The present disclosure describes compositions, isolated nucleic acids, fusion products, pharmaceutical compositions, and methods of using the disclosed compositions, isolated nucleic acids, fusion products, pharmaceutical compositions thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, vectors, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “approximately” or “about,” as applied to one or more values of interest, refers to a value that is similar to a stated reference value. In an aspect, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%. 2%, 1%, or less in either direction of the stated reference value unless otherwise stated or otherwise evident from the context.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration. In an aspect, a subject can be a human being. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have stressed, damaged, and/or injured tissues, or be suspected of having stressed, damaged, and/or injured tissues, or be at risk of developing stressed, damaged, and/or injured tissues.

As used herein, “effective amount” and “amount effective” can refer to an amount that is sufficient to achieve the desired result such as, for example, the treatment and/or regeneration of stressed, damaged, and/or injured tissues. As used herein, the terms “effective amount” and “amount effective” can refer to an amount that is sufficient to achieve the desired effect on stressed, damaged, and/or injured tissues. For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed composition that (i) treats the stressed, damaged, and/or injured tissues, (ii) attenuates, ameliorates, or eliminates one or more symptoms of the particular stressed, damaged, and/or injured tissues, or (iii) delays the onset of one or more symptoms of the particular stressed, damaged, and/or injured tissues described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In an aspect, a preparation can be administered in a “prophylactically effective amount”; that is, an amount effective for prevention of a disease, a disorder, an infection, a symptom, and/or a complication.

“Control” as used herein refers a standard or reference condition, against which results are compared. In an aspect, a control is used at the same time as a test variable or subject to provide a comparison. In an aspect, a control is a historical control that has been performed previously, a result or amount that has been previously known, or an otherwise existing record. A control may be a positive or negative control.

As used herein, probiotics refer to live microbial agents that promote the ecological balance of the intestinal flora and have beneficial effects on the host. Common probiotics mainly include Iwo main groups: bifidobacteria (bifidobacteria) and lactobacilli (lactobacilli), respectively. Ingestion of a suitable amount of probiotic bacteria can produce one or more specific functional health benefits to the host and therefore, probiotic bacteria have broad application prospects in functional foods and pharmaceuticals.

As used herein, prebiotics are food ingredients that are not readily digestible and that can beneficially affect the host by selectively simulating the growth and activity of one or more bacteria, thereby improving host health. Prebiotics can be divided into two categories based on chemical structure: ITF inulin-type fructans and GOS galacto-oligosaccharides, most of the commercially used prebiotics are oligosaccharides such as isomalto-oligosaccharides, fructo-oligosaccharides, galacto-oligosaccharides, etc.

As used herein, the term “diagnosed” means having been subjected to a physical examination by a person of skill, for example, a physician, and found to have stressed, damaged, and/or injured tissues that can be diagnosed or treated by one or more of the disclosed purified EPSs, the disclosed nucleic acids, the disclosed vectors, the disclosed compositions, the disclosed pharmaceutical preparations, and/or the disclosed methods. For example, “suspected of having” can mean having been subjected to a physical examination by a person of skill, for example, a physician, and found to have stressed, damaged, and/or injured tissues that can likely be treated by one or more of the disclosed purified EPSs, the disclosed nucleic acids, the disclosed vectors, the disclosed compositions, the disclosed pharmaceutical preparations, and/or the disclosed methods.

The words “treat” or “treating” or “treatment” refer to therapeutic or medical treatment wherein the object is to slow down (lessen), ameliorate, and/or diminish an undesired physiological change, disease, disorder, injury, pathological condition, or disorder in a subject. As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete clearance of an infection but may reduce or minimize complications, the side effects, and/or the progression of a disease, a disorder, an injury, an infection, a symptom, and/or a complication. The success or otherwise of treatment may be monitored by physical examination of the subject as well as cytopathological, DNA, and/or mRNA detection techniques. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the undesired physiological change, disease, injury, insult, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating stressed, damaged, and/or injured tissues can reduce the severity of stressed, damaged, and/or injured tissues in a subject by 1%-100% as compared to a control (such as, for example, a subject not having the disease, the disorder, the injury, the infection, the symptom, and/or the complication). In an aspect, treating can refer to a 1%, 2%, 3%, 4%, 5%, 6%, 7%, 8%, 9% 10%, 20%, 30%, 40%, 50%, 60%, 70%, 80%, 90%, or 100% reduction in the severity of stressed, damaged, and/or injured tissues. In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms. It is understood that treatment does not necessarily refer to a cure or complete ablation or eradication of the stressed, damaged, and/or injured tissues. However, in an aspect, treatment can refer to a cure or complete ablation or eradication of the stressed, damaged, and/or injured tissues.

A “patient” refers to a subject afflicted with stressed, damaged, and/or injured tissues. In an aspect, a patient can refer to a subject that has been diagnosed with or is suspected of having a disease, disorder, infection, symptom, and/or complication that results in stressed, damaged, and/or injured tissues. In an aspect, a patient can refer to a subject that has been diagnosed with or is suspected of having an established disease, disorder, infection, symptom, and/or complication that results in stressed, damaged, and/or injured tissues and is seeking treatment or receiving treatment.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease, disorder, infection, symptom, and/or complication is intended. In an aspect, preventing stressed, damaged, and/or injured tissues is intended. The words “prevent” and “preventing” and “prevention” also refer to prophylactic or preventative measures for protecting or precluding a subject (e.g., an individual) not having a given complication associated with stressed, damaged, and/or injured tissues from progressing to that complication.

As used herein, the terms “administering” and “administration” refer to any method of providing one or more of the disclosed purified EPSs, the disclosed isolated nucleic acid molecules, disclosed pharmaceutical formulations, disclosed vectors, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed nucleic acid molecule, a disclosed vector, a disclosed therapeutic agent, a disclosed pharmaceutical formulation, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

As used herein, “concurrently” means (1) simultaneously in time, or (2) at different times during the course of a common treatment schedule.

The term “contacting” as used herein refers to bringing one or more of the disclosed purified EPSs, the disclosed nucleic acids, the disclosed vectors, the disclosed compositions, and/or the disclosed pharmaceutical compositions together with a target area or intended target area (i.e., stressed, damaged, and/or injured tissues) in such a manner that the one or more disclosed purified EPSs, disclosed nucleic acids, disclosed vectors, disclosed compositions, and/or disclosed pharmaceutical formulation can exert an effect on the intended target or targeted area (i.e., stressed, damaged, and/or injured tissues) either directly or indirectly.

As used herein, “determining” can refer to measuring or ascertaining the presence and severity of a disease, disorder, injury, infection, symptom, and/or complication or the presence and severity of stressed, damaged, and/or injured tissues. Methods and techniques used to determining the presence and/or severity of a disease, disorder, injury, infection, symptom, and/or complication or the presence and/or severity of stressed, damaged, and/or injured tissues are typically known to the medical arts. For example, the art is familiar with the ways to identify and/or diagnose the presence, severity, or both of a disease, disorder, infection, symptom, and/or complication, or the presence, severity, or both of stressed, damaged, and/or injured tissues.

As used herein, an “isolated” biological component (such as a nucleic acid molecule, protein, or virus) has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components.

As used herein, the term “ILC3” refers to group 3 innate lymphoid cells.

The term “disease, disorder and/or physiological condition” as used herein includes, but is not limited to, any abnormal condition and/or disorder of a structure or a function that affects a part of an organism. It may be caused by an external factor, such as an infectious disease, or by internal dysfunctions, such as cancer, congenital condition, autoimmune disorder, genetic mutation, and the like.

As used herein, the term “ILC3-mediated disease” refers to any disease that results from or is in part mediated by, the presence of, activation of, upregulation of, participation of, absence of, de-activation of, or down-regulation of, ILC3 cells. Examples include, but are not limited to, inflammatory bowel disease (e.g., Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease), psoriasis, asthma, allergy-induced asthma, obesity, and the like.

### B. Agents

### 1. Biologically Active Agents

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

### 2. Pharmaceutically Active Agents

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

### 3. Anti-Bacterial Agents

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. For example, as used herein, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile). Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide, and periodate), or a combination thereof. As used herein, the recitation of an anti-bacterial agent inherently encompasses the pharmaceutically acceptable salts thereof.

### 4. Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents. For example, as used herein, an anti-fungal agent can comprise Abafungin, Albaconazole, Amorolfin, Amphotericin B, Anidulafungin. Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial. Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. As used herein, the recitation of an anti-fungal agent inherently encompasses the pharmaceutically acceptable salts thereof.

### 5. Anti-Viral Agents

Anti-viral agents are known to the art. As used herein, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc. Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine). Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. As used herein, the recitation of any anti-viral agent inherently encompasses the pharmaceutically acceptable salts thereof.

### 6. Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. As used herein, the recitation of a corticosteroid inherently encompasses the pharmaceutically acceptable salts thereof.

### 7. Analgesics

The compositions of the present disclosure can also be used in combination therapies with opioids and other analgesics, including narcotic analgesics, Mu receptor antagonists. Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods described herein with one or more compounds selected from aceclofenac, acemetacin, .alpha.-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis (acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, .alpha.-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethvlmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof

### 8. Immunostimulants

The term “immunostimulant” is used herein to describe a substance which evokes, increases, and/or prolongs an immune response to an antigen. Immunomodulatory agents modulate the immune system, and, as used herein, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators. Immunostimulants can include, but are not limited to, levamisole, thalidomide, erythema nodosum leprosum, BCG, cytokines such as interleukins or interferons, including recombinant cytokines and interleukin 2 (aldeslukin), 3D-MPL, QS21, CpG ODN 7909, miltefosine, anti-PD-1 or PD-1 targeting drugs, and acid (DCA, a macrophage stimulator), imiquimod and resiquimod (which activate immune cells through the toll-like receptor 7), chlorooxygen compounds such as tetrachlorodecaoxide (TCDO), agonistic CD40 antibodies, soluble CD40L, 4-1BB:4-1BBL agonists, OX40 agonists, TLR agonists, moieties that deplete regulatory T cells, arabinitol-ceramide, glycerol-ceramide, 6-deoxy and 6-sulfono-myo-insitolceramide, iNKT agonists, and TLR agonists. As used herein, the recitation of an immunostimulant inherently encompasses the pharmaceutically acceptable salts thereof.

### 9. Immune-Based Product

As used herein, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. As used herein, the recitation of an immune-based product inherently encompasses the pharmaceutically acceptable salts thereof.

### C. Compositions

### 1. Purified Exopolysaccharide (EPS)

Disclosed herein is One aspect of the present disclosure provides a purified exopolysaccharide (EPS) isolated from C. immunis, said EPS comprising, consisting or, or consisting essentially of a plurality of mannose and glucose subunits and a phosphocholine.

In an e embodiment, the phosphocholine is present at 3.22 ppm on a ¹H-NMR spectra. In an aspect, the purified EPS can comprise peaks at 160, 88, 35, 26, and 5 kDa when analyzed for distribution size through size exclusion chromatography (SEC).

In an aspect, the purified EPS has a mannan-like structure, wherein the backbone of the EPS predominantly can comprise mannose.

In an aspect, the purified EPS can comprise correlation spectroscopy (COSY) spectra showing a cross peak between the phosphocholine protons at 4.33 and 3.65 ppm.

### 2. Pharmaceutical Compositions

In another aspect, the present disclosure provides compositions comprising one or more of the compounds as described herein (e.g., a purified EPS) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent a disease, such as an ILC3-mediated disease, the compounds described herein may be administered singly, as mixtures of one or more compounds or in mixture or combination with other agents (e.g., therapeutic agents) useful for treating such diseases and/or the symptoms associated with such diseases. Such agents may include, but are not limited to, antibiotics, NSAIDS, anti-inflammatory compounds, cell therapy, immunomodulators, steroids/corticosteroids, polyclonal or monoclonal antibodies, non-immune tolerizing approaches, exosome therapy, and faecal transplants, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, tale or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

A disclosed pharmaceutically acceptable carrier can refer to a sterile aqueous or nonaqueous solution, a dispersion, a suspension, an emulsion, or any combination thereof, as well as a sterile powder for reconstitution into a sterile injectable solution, dispersion, suspension, emulsion, or any combination thereof just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

### 3. Food Composition

In another aspect, the present disclosure provides compositions comprising one or more of the compounds as described herein (e.g., a purified EPS) as a food additive, a food composition (particularly, a functional food composition), a feed additive or the like depending on the intended use or aspect. In addition, the content of purified EPS as an active ingredient can be also be adjusted within a wide range depending on the specific type, intended use or aspect of the food additive or food composition.

Furthermore, the content of the purified EPS as an active ingredient in the food composition according to aspects of the present invention can comprise 0.01 to 99 wt %, preferably 0.1 to 50 wt %, more preferably 0.5 to 25 wt %, based on the total weight of the composition, but is not limited thereto. The food composition according to embodiments of the present invention may be in the form of pellets, powders, granules, infusions, tablets, capsules, liquid or the like, and specific examples of the food may include meats, breads, chocolates, candies, snacks, confectionaries, gums, dairy products including ice creams, various kinds of soups, beverages, teas, functional water, drinks, alcoholic beverages, vitamin complexes and the like, and may include all health foods in a general sense. The food composition of the present invention may further contain sitologically acceptable carriers, various flavoring agents or natural carbohydrates as additional ingredients, in addition to the active ingredient. Additionally, the food composition of the present invention may contain various nutrients, vitamins, electrolytes, flavoring agents, coloring agents, pectic acid and its salt, alginic acid and its salt, an organic acid, a protective colloidal thickener, a pH adjusting agent, a stabilizer, a preservative, glycerin, alcohol, a carbonating agent used for carbonated drinks and the like. Additionally, the food composition of the present invention may contain fruit flesh for preparing natural fruit juices, fruit juice drinks and vegetable drinks. These ingredients may be used independently or as a mixture. The above-described natural carbohydrates may include monosaccharides such as glucose and fructose, disaccharides such as maltose and sucrose, polysaccharides such as dextrin and cyclodextrin and sugar alcohols such as xylitol, sorbitol, and erythritol. As a flavoring agent, a natural flavoring agent such as thaumatin or a stevia extract, or a synthetic flavoring agent such as saccharin or aspartame may be used.

### D. Methods

The purified EPS compounds of the present disclosure may be used in a number of methods for the regulation of genes and/or treatment and/or prevention of diseases, such as ILC3-mediated diseases.

1. Methods of Treating and/or Preventing Diseases and/or Disorders
(a) Methods of Reducing the Amount of, Activity of, and/or Proliferation of Colonic Group 3 Innate Lymphoid Cells (ILC3s)

Accordingly, an aspect of the present disclosure provides a method of reducing the amount of, activity of, and/or proliferation of colonic group 3 innate lymphoid cells (ILC3s) in a subject, the method comprising, consisting of, or consisting essentially of administering to the subject a therapeutically effective amount of a disclosed purified EPS, or a pharmaceutical composition thereof, such that the amount of, activity of, and/or proliferation of ILC3 cells are reduced in the subject.

In an aspect, a method can further comprise administering to the subject that weighs an average of 70 kg a daily dose of between about 1 g and about 70 g of the disclosed purified EPS, of a pharmaceutical composition thereof; more preferably between about 5 g and about 60 g of the disclosed purified EPS, of a pharmaceutical composition thereof; and most preferably between about 10 g and about 50 g of the disclosed purified EPS, of a pharmaceutical composition thereof.

In an embodiment, a disclosed method can further comprise administering to the subject one or more additional therapeutic agents. In another aspect, a disclosed method can further comprise co-administering to the subject one or more additional therapeutic agents prior to, concurrently with, or after the administration of a disclosed purified EPS or a disclosed pharmaceutical composition thereof.

In an embodiment, the disclosed one or more additional therapeutic agents can be administered prior to a disclosed purified EPS or a disclosed pharmaceutical composition thereof. In another embodiment, the one or more additional therapeutic agents can be administered concurrently with a disclosed purified EPS or a disclosed pharmaceutical composition thereof.

In another embodiment, the one or more additional therapeutic agents can be administered after a disclosed purified EPS or a disclosed pharmaceutical composition thereof.

In an aspect, a disclosed method can further comprise diagnosing a subject as having a need for reducing the amount of, activity of, and/or proliferation of colonic group 3 innate lymphoid cells (ILC3s).

In an aspect, a disclosed method can further comprise titrating the dose of a disclosed purified EPS or a disclosed pharmaceutical composition. In an aspect of a disclosed method, a therapeutically effective amount can comprise the maximum tolerated dose of a disclosed purified EPS or a disclosed pharmaceutical.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a disclosed method can be altered by changing the amount of one or more of a disclosed purified EPS or a disclosed pharmaceutical composition, one or more of the disclosed isolated nucleic acid molecules, one or more disclosed vectors, or any combination thereof administered to a subject, or by changing the frequency of administration of one or more of a disclosed purified EPS or a disclosed pharmaceutical composition, one or more of the disclosed isolated nucleic acid molecules, one or more disclosed vectors, or any combination thereof, or any combination thereof to a subject, or by changing the duration of time one or more of a disclosed purified EPS or a disclosed pharmaceutical composition, one or more of the disclosed isolated nucleic acid molecules, one or more disclosed vectors, or any combination thereof are administered to a subject.

In an aspect, a disclosed method can further comprise administering one or more times a disclosed purified EPS or a disclosed pharmaceutical composition, a disclosed isolated nucleic acid molecule, a disclosed vector, a disclosed pharmaceutical composition, or any combination thereof.

In an aspect, a disclosed method can further comprise monitoring the subject for adverse effects. In an aspect, in the absence of adverse effects, a disclosed method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

### (b) Methods of Treating or Preventing an ILC3-Mediated Disease

Another aspect of the present disclosure provides a method of treating or preventing an ILC3-mediated disease in a subject, the method comprising, consisting of, or consisting essentially of administering to the subject a therapeutically effective amount of a disclosed purified EPS, or a disclosed pharmaceutical composition thereof, such that the ILC3-mediated disease is treated and/or prevented in the subject.

In an aspect, a method can further comprise administering to the subject that weighs an average of 70 kg a daily dose of between about 1 g and about 70 g of the disclosed purified EPS, of a pharmaceutical composition thereof; more preferably between about 5 g and about 60 g of the disclosed purified EPS, of a pharmaceutical composition thereof; and most preferably between about 10 g and about 50 g of the disclosed purified EPS, of a pharmaceutical composition thereof.

In some embodiments, the ILC3-mediated disease is selected from the group consisting of inflammatory bowel disease, psoriasis, asthma, allergy-induced asthma, obesity, multiple sclerosis, enteric infections, necrotizing enterocolitis, MetS, and combinations thereof.

In an embodiment, the ILC3-mediated disease can comprise obesity.

In an embodiment, the ILC3-mediated disease can comprise IBD. In an embodiment, the IBD can be selected from the group consisting of Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease, Behcet's disease and combinations thereof.

In an embodiment, a disclosed method can further comprise administering to the subject one or more additional therapeutic agents. In another aspect, a disclosed method can further comprise co-administering to the subject one or more additional therapeutic agents prior to, concurrently with, or after the administration of the purified EPS or a pharmaceutical composition thereof.

In an embodiment, a disclosed one or more additional therapeutic agents can be administered prior to the purified EPS or pharmaceutical composition thereof. In another embodiment, a disclosed one or more additional therapeutic agents can be administered concurrently with the purified EPS or pharmaceutical composition thereof.

In another embodiment, the one or more additional therapeutic agents is administered after the purified EPS or pharmaceutical composition thereof.

In an aspect, a disclosed method can further comprise diagnosing a subject having a ILC3-mediated disease.

In an aspect, a disclosed method can further comprise titrating the dose of a disclosed purified EPS or a disclosed pharmaceutical composition. In an aspect of a disclosed method, a therapeutically effective amount can comprise the maximum tolerated dose of a disclosed purified EPS or a disclosed pharmaceutical.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a disclosed method can be altered by changing the amount of one or more of a disclosed purified EPS or a disclosed pharmaceutical composition, one or more of the disclosed isolated nucleic acid molecules, one or more disclosed vectors, or any combination thereof administered to a subject, or by changing the frequency of administration of one or more of a disclosed purified EPS or a disclosed pharmaceutical composition, one or more of the disclosed isolated nucleic acid molecules, one or more disclosed vectors, or any combination thereof, or any combination thereof to a subject, or by changing the duration of time one or more of a disclosed purified EPS or a disclosed pharmaceutical composition, one or more of the disclosed isolated nucleic acid molecules, one or more disclosed vectors, or any combination thereof are administered to a subject.

In an aspect, a disclosed method can further comprise administering one or more times a disclosed purified EPS or a disclosed pharmaceutical composition, a disclosed isolated nucleic acid molecule, a disclosed vector, a disclosed pharmaceutical composition, or any combination thereof.

In an aspect, a disclosed method can further comprise monitoring the subject for adverse effects. In an aspect, in the absence of adverse effects, a disclosed method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

### (c) Methods of Decreasing Visceral Adiposity

Another aspect of the present disclosure provides a method of decreasing visceral adiposity in a subject, the method comprising, consisting of, or consisting essentially of administering to the subject a therapeutically effective amount of a purified EPS as provided herein, or a pharmaceutical composition thereof, such that the visceral adiposity is decreased in the subject.

Another aspect of the present disclosure provides a method of decreasing visceral fat in a subject, the method comprising, consisting of, or consisting essentially of administering to the subject a therapeutically effective amount of a purified EPS, or a pharmaceutical composition thereof, such that the visceral fat is decreased in the subject.

In an aspect, a method can further comprise administering to the subject that weighs an average of 70 kg a daily dose of between about 1 g and about 70 g of the disclosed purified EPS, of a pharmaceutical composition thereof; more preferably between about 5 g and about 60 g of the disclosed purified EPS, of a pharmaceutical composition thereof; and most preferably between about 10 g and about 50 g of the disclosed purified EPS, of a pharmaceutical composition thereof.

In an embodiment, a disclosed method can further comprise administering to the subject one or more additional therapeutic agents. In another aspect, a disclosed method can further comprise co-administering to the subject one or more additional therapeutic agents prior to, concurrently with, or after the administration of the purified EPS or a pharmaceutical composition thereof.

In an embodiment, the disclosed one or more additional therapeutic agents can be administered prior to the purified EPS or pharmaceutical composition thereof. In another embodiment, the disclosed one or more additional therapeutic agents can be administered concurrently with the purified EPS or pharmaceutical composition thereof.

In another embodiment, the one or more additional therapeutic agents can be administered after the purified EPS or pharmaceutical composition thereof.

In an aspect, a disclosed method can further comprise diagnosing a subject having visceral adiposity.

In an aspect, a disclosed method can further comprise titrating the dose of a disclosed purified EPS or a disclosed pharmaceutical composition. In an aspect of a disclosed method, a therapeutically effective amount can comprise the maximum tolerated dose of a disclosed purified EPS or a disclosed pharmaceutical.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a disclosed method can be altered by changing the amount of one or more of a disclosed purified EPS or a disclosed pharmaceutical composition, one or more of the disclosed isolated nucleic acid molecules, one or more disclosed vectors, or any combination thereof administered to a subject, or by changing the frequency of administration of one or more of a disclosed purified EPS or a disclosed pharmaceutical composition, one or more of the disclosed isolated nucleic acid molecules, one or more disclosed vectors, or any combination thereof, or any combination thereof to a subject, or by changing the duration of time one or more of a disclosed purified EPS or a disclosed pharmaceutical composition, one or more of the disclosed isolated nucleic acid molecules, one or more disclosed vectors, or any combination thereof are administered to a subject.

In an aspect, a disclosed method can further comprise administering one or more times a disclosed purified EPS or a disclosed pharmaceutical composition, a disclosed isolated nucleic acid molecule, a disclosed vector, a disclosed pharmaceutical composition, or any combination thereof.

In an aspect, a disclosed method can further comprise monitoring the subject for adverse effects. In an aspect, in the absence of adverse effects, a disclosed method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

2. Methods of Inhibiting and/or Decreasing the Expression of a Gene

Another aspect of the present disclosure provides a method of inhibiting and/or decreasing the expression of a gene selected from the group consisting of Cd36, Scd1, Nfil3 and any target genes thereof, in a cell, the method comprising, consisting of, or consisting essentially of contacting the cell with an effective amount of a purified EPS as provided herein such that the expression of the gene and/or any target genes thereof are decreased and/or inhibited in the cell.

Another aspect of the present disclosure provides a method of inhibiting and/or decreasing the expression of a gene selected from the group consisting of Cd36, Scd1, Nfil3 and any target genes thereof, in a subject, the method comprising, consisting of, or consisting essentially of administering to the subject a therapeutically effective amount of a purified EPS as provided herein, or a pharmaceutical composition thereof, such that the expression of the gene and/or any target genes thereof are decreased and/or inhibited in the subject.

Another aspect of the present disclosure provides a method of increasing the expression of the gene Csf2 and any target genes thereof, in a cell, the method comprising, consisting of, or consisting essentially of contacting the cell with an effective amount of a purified EPS as provided herein such that the expression of the Csf2 gene and/or any target genes thereof are increased in the cell.

Another aspect of the present disclosure provides a method of increasing the expression of the gene Csf2, and any target genes thereof, in a subject, the method comprising, consisting of, or consisting essentially of administering to the subject a therapeutically effective amount of a purified EPS as provided herein, or a pharmaceutical composition thereof, such that the expression of the Csf2 gene and/or any target genes thereof are increased in the subject.

### E. Co-Administration of Additional Therapeutic Agent(s)

In an embodiment, a disclosed method can further comprise administering to the subject one or more additional therapeutic agents. In an embodiment, the disclosed one or more additional therapeutic agent(s) can be administered prior to the purified EPS as provided herein or pharmaceutical composition thereof. In another embodiment, the disclosed one or more additional therapeutic agent(s) can be administered concurrently with the purified EPS as provided herein or pharmaceutical composition thereof. In an embodiment, the disclosed one or more additional therapeutic agent(s) can be administered after the purified EPS as provided herein or pharmaceutical composition thereof.

In an aspect, patients in need of reduction of the amount of, activity of, and/or proliferation of colonic group 3 innate lymphoid cells (ILC3s), in need of treatment for or prevention of an ILC3-mediated disease, decreased visceral adiposity, inhibition and/or in need of decrease of the expression of a gene selected from the group consisting of Cd36, Scd1, Nfil3, and/or increase of the expression of the gene Csf2 and any target genes thereof, may have diseases or conditions associated with the gastrointestinal tract that can benefit from a combination treatment. In an aspect, such combination treatment can comprise a therapeutically effective amount of a purified EPS as provided herein, or a pharmaceutical composition thereof and one or more additional therapeutic agent(s) to treat gastrointestinal disorders. More specifically, therapeutic agents used to treat gastrointestinal disorders may include proton pump inhibitors, such as pantoprazole (Protonix), lansoprazole (Prevacid), esomeprazole (Nexium), omeprazole (Prilosec), rabeprazole; H2 blockers, such as cimetidine (Tagamet), ranitidine (Zantac), famotidine (Pepcid), nizatidine (Axid); prostaglandins, such as misoprostoL (Cytotec); sucralfate; and antacids. In another aspect, one or more disclosed antiacids help prevent modification of the purified EPS as provided herein, or a pharmaceutical composition thereof.

In another aspect, the Patient may benefit from a combination treatment of a therapeutically effective amount of a purified EPS as provided herein, or a pharmaceutical composition thereof and one or more additional therapeutic agents to lower the amounts of cholesterol or fatty sugars present in the blood. Some examples of lipid lowering agents include bezafibrate (Bezalip), ciprofibrate (Modalim), and statins, such as atorvastatin (Lipitor), fluvastatin (Lescol), lovastatin (Mevacor, Altocor), mevastatin, pitavastatin (Livalo, Pitava) pravastatin (Lipostat), rosuvastatin (Crestor), and simvastatin (Zocor).

In another aspect the Patient may benefit from a combination treatment of a therapeutically effective amount of a purified EPS as provided herein, or a pharmaceutical composition thereof and one or more additional therapeutic agents to treat medical conditions such as one or more of a metabolic disorder, a pulmonary disorder, a peripheral vascular disorder, cardiovascular related disorder, and cancer, e.g., colon cancer.

Examples of metabolic disorders include, without limitation, diabetes, including type I and type II diabetes, dyslipidemia, glucose intolerance, hypertension, elevated serum cholesterol, and elevated triglycerides.

Examples of therapeutic agents used to treat metabolic disorders include antihypertensive agents and lipid lowering agents, as described above. Some examples of additional therapeutic agents used to treat metabolic disorders include insulin, sulfonylureas, biguanides, alpha-glucosidase inhibitors, and incretin mimetics.

Pulmonary disorder refers to any disease or condition related to the lungs. Examples of pulmonary disorders include, without limitation, asthma, chronic obstructive pulmonary disease (COPD), bronchitis, and emphysema.

Examples of therapeutics agents used to treat pulmonary disorders include bronchodilators including beta2 agonists and anticholinergics, corticosteroids, and electrolyte supplements. Specific examples of therapeutic agents used to treat pulmonary disorders include epinephrine, terbutaline (Brethaire, Bricanyl), albuterol (Proventil), salmeterol (Serevent, Serevent Diskus), theophylline, ipratropium bromide (Atrovent), tiotropium (Spiriva), methylprednisolone (Solo-Medrol, Medrol), magnesium, and potassium.

Peripheral vascular disorders are disorders related to the blood vessels (arteries and veins) located outside the heart and brain, including, for example peripheral arterial disease (PAD), a condition that develops when the arteries that supply blood to the internal organs, arms, and legs become completely or partially blocked as a result of atherosclerosis.

In an aspect, the Patient may exhibit conditions that benefit from administration of one or more therapeutic agent or agents that are antibiotics, analgesics, antidepressant and anti-anxiety agents.

Antidepressant and anti-anxiety agents include those agents used to treat anxiety disorders, depression, and those used as sedatives and tranquillers. Examples of antidepressant and anti-anxiety agents include benzodiazepines, such as diazepam, lorazepam, and midazolam; enzodiazepines; barbiturates; glutethitnide; chloral hydrate; meprobamate; sertraline (Zoloft, Lustral, Apo-Sertral, Asentra, Gladern, Serlift, Stimuloton); escitalopram (Lexapro, Cipralex); fluoxetine (Prozac, Sarafem, Fluctin, Fontex, Prodep, Fludep, Lovan); venlafaxine (Effexor XR, Efexor); citalopram (Celexa, Cipramil, Talohexane); paroxetine (Paxil, Seroxat, Aropax); trazodone (Desyrel); amitriptyline (Elavil); and bupropion (Wellbutrin, Zyban).

Cardiovascular related diseases or conditions that may benefit from a combination treatment of a therapeutically effective amount of a purified EPS as provided herein, or a pharmaceutical composition thereof and one or more additional therapeutic agents include, without limitation, angina, including stable angina, unstable angina (UA), exercised-induced angina, variant angina, arrhythmias, intermittent claudication, myocardial infaretion including non-STE myocardial infaretion (NSTEMI), heart failure including congestive (or chronic) heart failure, acute heart failure, or recurrent ischemia.

Therapeutic agents suitable for treating cardiovascular related diseases or conditions include anti-anginals, heart failure agents, antithrombotic agents, antiarrhythmic agents, antihypertensive agents, and lipid lowering agents.

Anti-anginals include beta-blockers, calcium channel blockers, and nitrates. Beta blockers reduce the heart's need for oxygen by reducing its workload resulting in a decreased heart rate and less vigorous heart contraction. Nitrates dilate the arteries and veins thereby increasing coronary blood flow and decreasing blood pressure. Examples of nitrates include nitroglycerin, nitrate patches, isosorbide dinitrate, and isosorbide-5-mononitrate. Calcium channel blockers prevent the normal flow of calcium to the cells of the heart and blood vessels causing the blood vessels to relax thereby increasing the supply of blood and oxygen to the heart.

Agents used to treat heart failure include diuretics, ACE inhibitors, vasodilators, and cardiac glycosides. Diuretics eliminate excess fluids in the tissues and circulation thereby relieving many of the symptoms of heart failure.

Angiotensin converting enzyme (ACE) inhibitors reduce the workload on the heart by expanding the blood vessels and decreasing resistance to blood flow.

Vasodilators reduce pressure on the blood vessels by making them relax and expand.

Cardiac glycosides are compounds that increase the force of the heart's contractions. These compounds strengthen the pumping capacity of the heart and improve irregular heartbeat activity. Examples of cardiac glycosides include digitalis, digoxin, and digitoxin.

Antithrombotics inhibit the clotting ability of the blood. There are three main types of antithrombotics-platelet inhibitors, anticoagulants, and thrombolytic agents. Platelet inhibitors inhibit the clotting activity of platelets, thereby reducing clotting in the arteries. Anticoagulants prevent blood clots from growing larger and prevent the formation of new clots. Thrombolytic agents act to break down an existing blood clot. Antiarrhythmic agents are used to treat disorders of the heart rate and rhythm. Cardiac glycosides and beta blockers are also used as antiarrhythmic agents. Antihypertensive agents are used to treat hypertension, a condition in which the blood pressure is consistently higher than normal. Hypertension is associated with many aspects of cardiovascular disease, including congestive heart failure, atherosclerosis, and clot formation.

In another embodiment, the disclosed methods can further comprise administering to the subject a pre-biotic. In an embodiment, the prebiotic can be administered prior to the purified EPS as provided herein or pharmaceutical composition thereof. In another embodiment, a disclosed prebiotic can be administered concurrently with the purified EPS as provided herein or pharmaceutical composition thereof. In another embodiment, a disclosed prebiotic can be administered after the purified EPS as provided herein or pharmaceutical composition thereof.

In an aspect, the disclosed one or more prebiotic(s) can be administered to the Patient to enhance production of EPS by C. immunis naturally present in the Patient's digestive tract. For example, a prebiotic according to embodiments of the invention can be an effective amount of choline and/or mannose for enhanced production of a disclosed EPS can be administered.

In another aspect, C. immunis can be co-administered as a Probiotic with a disclosed purified EPS or a disclosed pharmaceutical composition thereof to have both the active molecule and an ongoing production source.

In an aspect, C. immunis can be co-administered as a Probiotic with, for example, choline and/or mannose for enhanced colonization and production of the EPS.

In another aspect, different gut commensal bacteria that contain the same gene locus (licABC), can be co-administered with C. immunis, as Probiotics, and/or the purified EPS.

### F. Kits

The present disclosure further provides kits comprising the compositions provided herein and for carrying out the subject methods as provided herein. For example, in an embodiment, a subject kit may comprise, consist of, or consist essentially of a disclosed purified EPS and/or a disclosed pharmaceutical composition.

In other embodiments, a disclosed kit may further include other components. Such components may be provided individually or in combinations, and may provide in any suitable container such as a vial, a bottle, or a tube. Examples of such components include, but are not limited to, one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g., in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### G. Methods to Isolate the Exopolysaccharide (EPS)

In an aspect, purified exopolysaccharide (EPS) from C. immunis is isolated in accordance with the following steps: culturing medium inoculated with C. immunis; centrifuging the cultured medium to pellet the C. immunis; filtering the supernatant; treating the filtered supernatant with DNaseI and RNaseA; centrifuging the DNaseI and RNaseA treated supernatant to clarify; treating the clarified supernatant with proteinase; dialyzing the proteinase treated supernatant by adding ultrapure water and spinning through a spin column to eliminate the proteinase; and precipitating the proteinase-free and dialyzed remaining solution with ethanol.

In an aspect, the culturing medium inoculated with C. immunis can be carried out for between about 2 hrs. and about 30 hrs., more preferably between about 5 hrs. and about 20 hrs., and most preferably between about 8 hrs. and about 15 hrs.

In an aspect, the filtering of the supernatant can be carried out through a 0.45 μm filter, and then through a 0.22 μm filter.

In an aspect the concentrating the supernatant can be carried out multiple times using a 100 kDa molecular weight cutoff spin column.

In an aspect, the treating the filtered supernatant with DNaseI and RNaseA can be carried out for between about 2 hrs. and about 30 hrs., more preferably between about 5 hrs. and about 20 hrs., and most preferably between about 8 hrs. and about 15 hrs.

In an aspect, the treating the clarified supernatant with proteinase can be carried out with proteinase K for between about 2 hrs. and about 30 hrs., more preferably between about 5 hrs. and about 20 hrs., and most preferably for between about 8 hrs. and about 15 hrs.

In an aspect, the dialyzing the proteinase treated supernatant by adding ultrapure water and spinning through a spin column to eliminate the proteinase can be carried out with a 100 kDa cutoff spin column.

In an aspect the adding ultrapure water and spinning through a spin column can be carried out multiple times.

In an aspect, the precipitating the proteinase-free and dialyzed remaining solution with ethanol can be carried out for between about 2 hrs. and about 30 hrs., more preferably for between about 5 hrs. and about 20 hrs., and most preferably for between about 8 hrs. and about 15 hrs.

In an aspect, a disclosed method further can comprise the step of quantifying the yield of purified EPS.

In an aspect, a disclosed method can further comprise validating the purity of the purified EPS. In an aspect, a disclosed method can further comprise evaluating the efficacy of a disclosed EPS. In an aspect, a disclosed method can further comprise evaluating the efficacy of a disclosed EPS in an established animal model and/or established cell model.

In an aspect, a disclosed method can further comprise cryopreserving the purified EPS. In an aspect, a disclosed method can further comprise thawing cryopreserved C. immunis to inoculate the medium.

### H. Plasmids

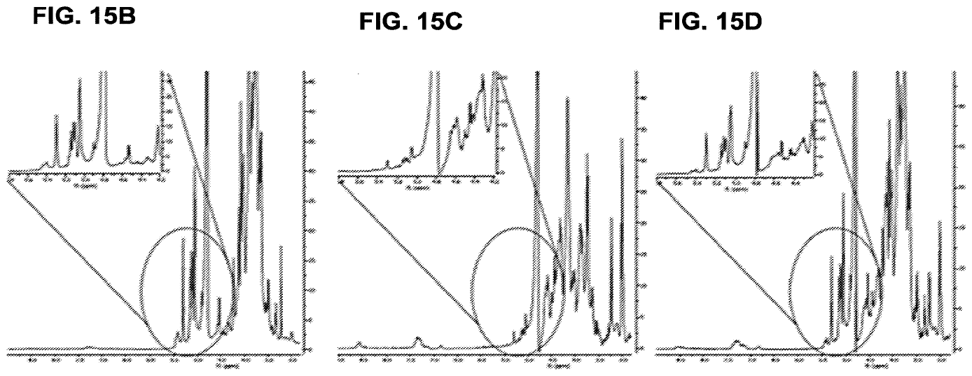

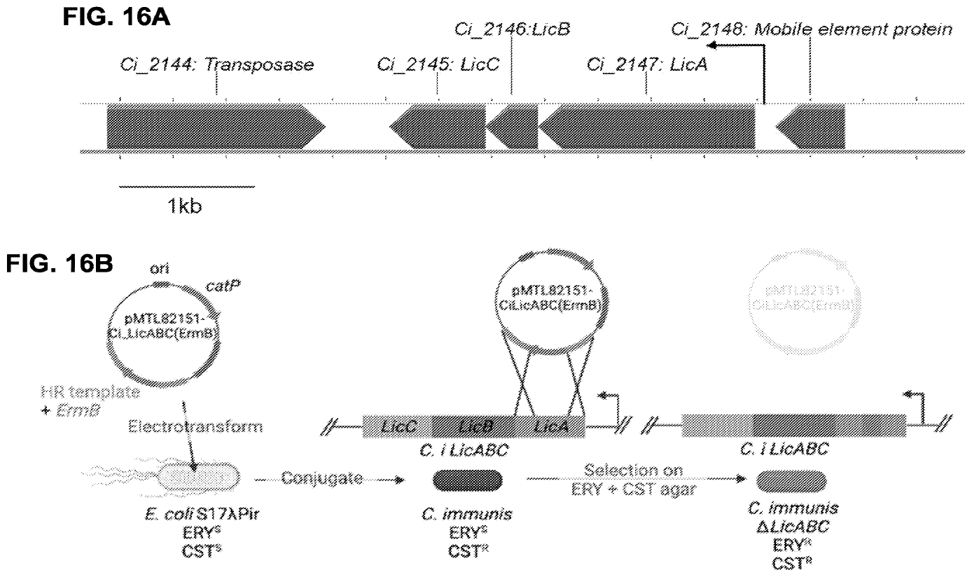

Disclosed herein is a plasmid comprising one or more disclosed isolated nucleic acid molecules. Disclosed herein is a plasmid comprising one or more disclosed vectors. Disclosed here are plasmids used in methods of making a disclosed composition such as, for example, a disclosed purified EPS, a disclosed enzyme, a disclosed recombinant enzyme, a disclosed isolated nucleic acid molecule, a disclosed vector, or a disclosed pharmaceutical formulation. Plasmids and using plasmids are known to the art. Disclosed herein is the plasmid depicted in FIG. 16B.

### I. Cells

Disclosed herein are cells comprising a disclosed isolated nucleic acid molecule, a disclosed vector, and/or a disclosed plasmid. Disclosed herein are cells transduced by one or more disclosed viral vectors. Disclosed herein are cells transduced by one or more disclosed recombinant viral vectors. Disclosed herein are cells transfected with one or more disclosed isolated nucleic acid molecules. In an aspect, a disclosed cell has been transfected or disclosed cells have been transfected with one or more disclosed nucleic acid sequences. In an aspect, a disclosed cell or disclosed cells can be humanized. Techniques to achieve transfection and transduction are known to the art and using transfected or transduced cells are known to the art. In an aspect, disclosed herein are human immortalized cells lines transduced by one or more disclosed viral vectors or transfected with one or more disclosed isolated nucleic acids or disclosed plasmids. In an aspect, disclosed herein are human immortalized cells lines contacted with one or more disclosed pharmaceutical formulations. Disclosed herein are cells obtained for a subject treated with one or more disclosed isolated nucleic acid molecule, one or more disclosed vectors, one or more disclosed plasmids, or one or more disclosed pharmaceutical formulations.

In an aspect, a disclosed cell or disclosed cells can be used to produce a disclosed EPS. In an aspect, a disclosed cell or disclosed cell used to produce a disclosed EPS can be a recombinant cell or recombinant cells. In an aspect, a disclosed recombinant cell can be engineered to produce more efficiently a disclosed EPS. In an aspect, disclosed cells can be prokaryotic cells or eukaryotic cells. In an aspect, a disclosed cell used in a disclosed method can comprise a recombinant bacterial cell. In an aspect, a disclosed cell used in a disclosed method can comprise a recombinant eukaryotic cell. Disclosed herein is a recombinant C. immunis cell or recombinant C. immunis cells. In an aspect, a disclosed recombinant C. immunis cell or recombinant C. immunis cells can be used in a disclosed method. Disclosed herein is a cryopreserved C. immunis cell or cryopreserved C. immunis cells. In an aspect, a disclosed cryopreserved cell or cryopreserved cells can be thawed and used in a disclosed method. Disclosed herein is a cryopreserved recombinant C. immunis cell or cryopreserved recombinant C. immunis cells. In an aspect, a disclosed cryopreserved recombinant C. immunis cell or cryopreserved recombinant C. immunis cells can be thawed and used in a disclosed method.

The following Examples are provided by way of illustration and not by way of limitation.

### VIII. EXAMPLES

### A. Example 1

### A Commensal-Derived Exopolysaccharide Regulates ILC3-Mediated Diseases

i. Abstract

The gut microbiota plays a fundamental role in controlling many facets of human physiology, with impacts on the immune system being chief among them. Although there have been increasing successes in identifying disease-regulating commensal bacteria, the mechanisms by which they work remain poorly understood. The inventors previously characterized Clostridium immunis, a new human-derived commensal bacterial species that modulates colitis. Here the immunological factors and specific bacterial determinant required for its disease modulating activity were interrogated. It was found that that C. immunis directly regulated the effector function of group 3 innate lymphoid cells (ILC3s), with altered levels of IL-22 and GM-CSF in states of health and inflammation, respectively. This impact on ILC3s serves as a common immunological node for modulation of visceral adiposity and intestinal inflammation. Surprisingly, the two bacterial species most closely related to C. immunis lack this ILC3-modulating activity. Guided by comparative genomics, an exopolysaccharide (EPS) was purified that recapitulates the immunomodulatory activity of C. immunis in vitro and in vivo. Structural analysis revealed that phosphocholine on the EPS correlated with activity, and deletion of the C. immunis phosphocholine biosynthesis locus (LicABC) resulted in functionally inactive EPS. The data demonstrated that a phosphocholine moiety present on a bacterial polysaccharide was required to orchestrate ILC3 effector function and regulate intestinal homeostasis. Considered together, the results shown herein provided genetic and structural insight into how a human commensal bacterium provides a unique immunomodulatory function.

ii. Results

The microbiota plays an integral role in regulating countless immune-mediated diseases, and there is a growing list of commensal bacteria causally linked to specific phenotypes. Given that taxonomically diverse commensal bacteria produce generally similar bioactive products (e.g., short-chain fatty acids, bile acids, canonical microbe-associated molecular patterns), it remains unclear, with limited exceptions, how particular organisms provide unique functions distinct from the other members of the microbiota. Ultimately, an improved understanding of the mechanisms that underlie host-microbiota interactions—from both the host and bacterial perspectives—will facilitate clarifying the genetic and structural determinants for this bacterial specificity.

The Immunological Determinants of C. immunis Activity:

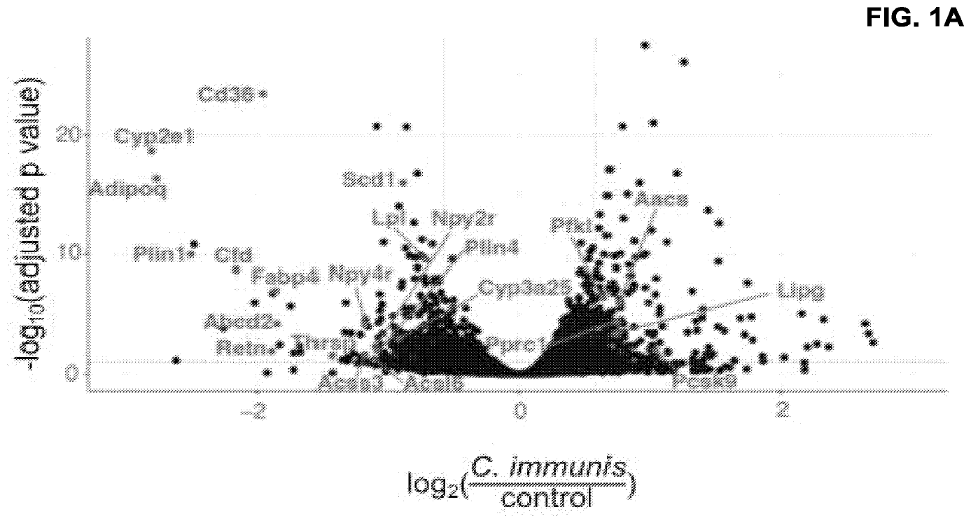

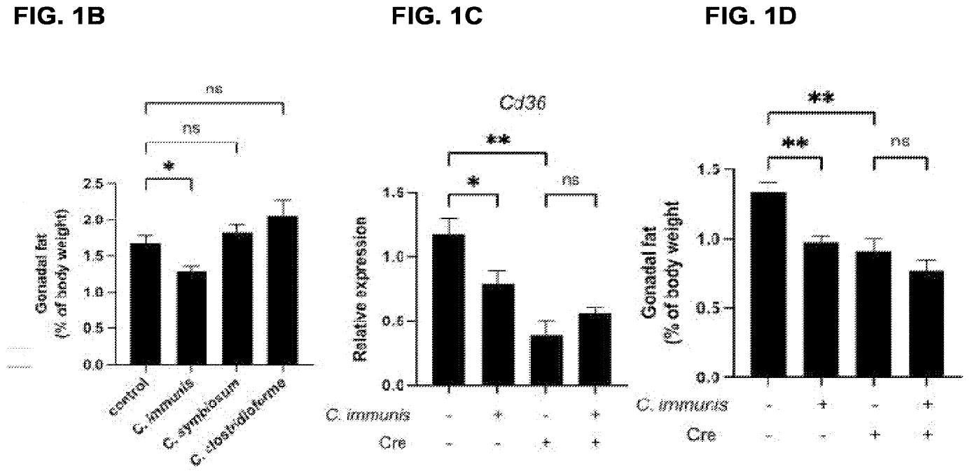

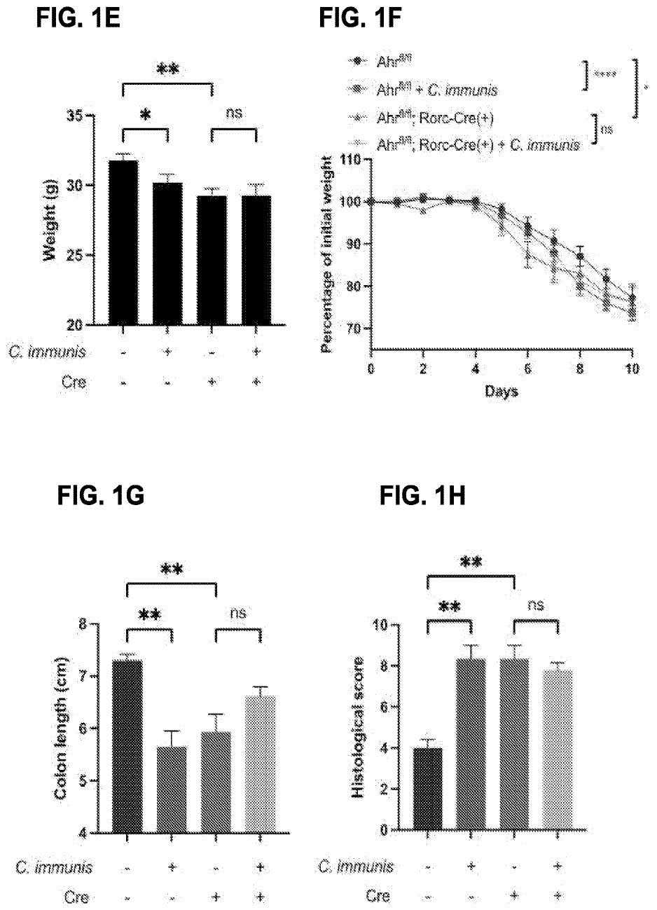

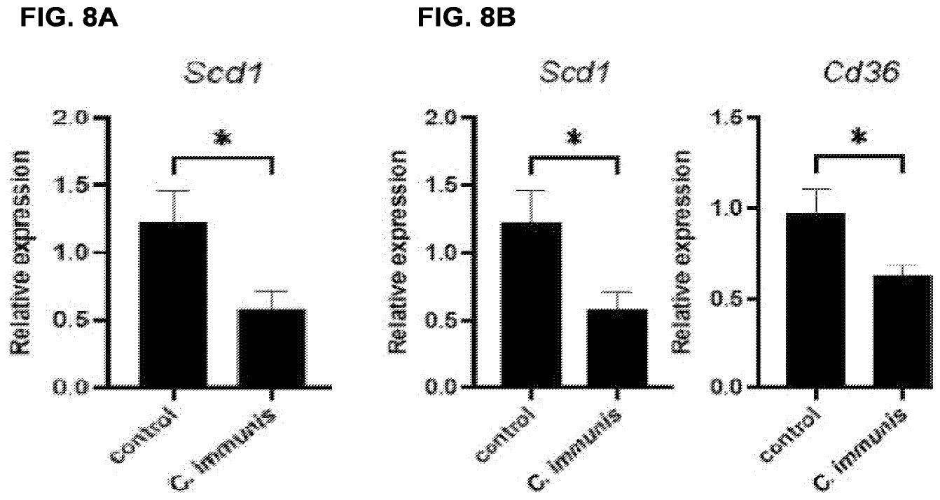

The present inventors recently characterized C. immunis, a newly described human-derived commensal bacterium that protects mice against colitis-associated death. To better understand how C. immunis protects against colitis, gnotobiotic mice harboring a mouse microbiota (MMb) with or without C. immunis were treated and compared their colonic transcriptional profile. Although no notable changes were detected in immune-related genes, it was observed that C. immunis treatment surprisingly led to suppression of genes related to lipid uptake and metabolism (FIG. 1A). These findings were confirmed to extend to the small intestine, the primary site of lipid metabolism, by demonstrating C. immunis inhibits small-intestinal expression of Cd36, which encodes a transporter that imports fatty acids into cells, and Scd1, which encodes a stearoyl-coenzyme A desaturase16 (FIG. 8). Given that deletion of either of these genes results in decreased body fat, it was reasoned that treatment with C. immunis may similarly reduce visceral adiposity. Indeed, MMb mice orally treated with C. immunis had less gonadal fat than untreated mice, which demonstrates that C. immunis decreases visceral adiposity (FIG. 1B). Intriguingly, neither Clostridium symbiosum nor Clostridium clostridioforme, the two bacterial species most closely related to C. immunis (genomic average nucleotide identity shared with C. immunis is 99% and 70%, respectively), had any impact on gonadal fat (FIG. 1B), a finding that indicates C. immunis has unique functionality compared to these related bacterial species. Although the microbiota generally promotes adiposity, treatment with C. immunis decreased visceral fat even in the presence of a complex microbiota.

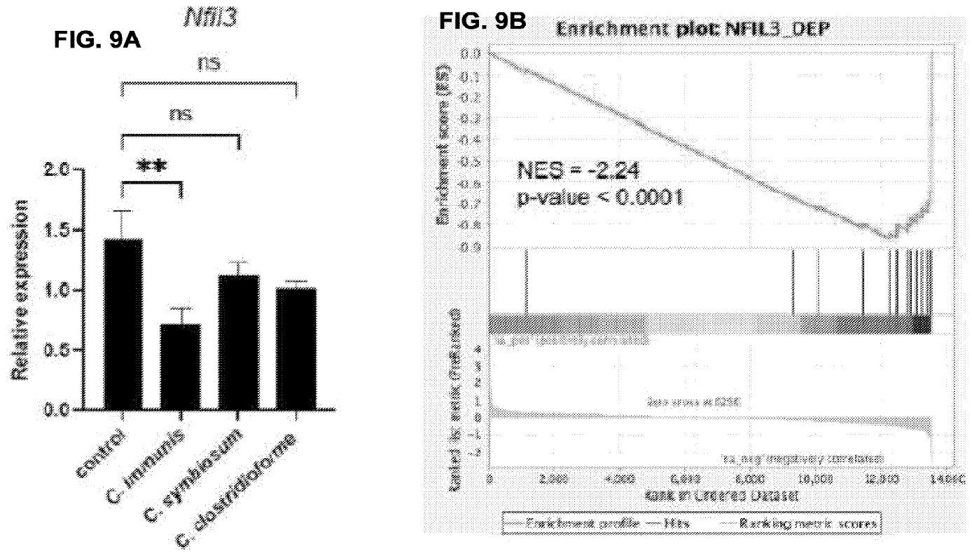

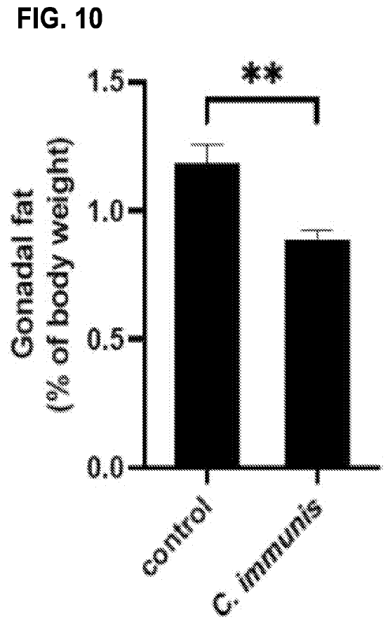

Recent work established that the microbiota positively regulates lipid metabolism genes and adiposity by increasing expression of the transcription factor NFIL3 through a pathway dependent on IL-22 secretion by group 3 innate lymphoid cells (ILC3s). Consistent with this model and the negative regulation of body fat by C. immunis, it was found that C. immunis decreased expression of Nfil3 and its target genes (FIG. 9), which showed that C. immunis may be working through this ILC3-IL-22-NFIL3 pathway. To investigate this further, ILC3-deficient mice were generated by breeding Ahrfl/fl mice with Rorc-cre mice, as previously described. These ILC3-deficient mice had decreased Cd36 expression, reduced gonadal fat, and lower body weights as compared to Ahrfl/fl littermate controls (FIG. 1C-FIG. 1E), findings that were consistent with the known role for ILC3s in obesity. While C. immunis decreased these measures in Ahrfl/fl mice, it had no effect in ILC3-deficient mice (FIG. 1C-FIG. 1E). Of note, the ILC3-deficient Ahrfl/fl Rorc-cre mice also lacked Ahr expression in T cells, which was implicated in the microbial regulation of body composition. The results were confirmed to not be related to T cells by showing C. immunis decreased visceral adiposity in Rag1/− mice (FIG. 10). Taken together, these data establish that C. immunis decreased visceral fat in an ILC3-dependent manner.

Since ILC3s also play a critical role in colitis, whether C. immunis similarly modulated colitis severity with ILC3s as the primary effector immune cell was examined. Here, ILC3-deficient mice were orally treated and their Ahrfl/fl littermates with or without C. immunis and subjected them to dextran sodium sulfate (DSS)-induced colitis. Ahrfl/fl mice treated with C. immunis phenocopied ILC3-deficient mice, with increased weight loss, shorter colons, and greater histological evidence of disease compared to untreated Ahrfl/fl mice (FIG. 1F-FIG. 1H). Importantly, C. immunis treatment did not impact disease in ILC3-deficient mice, thereby demonstrating C. immunis modulated colitis severity through an ILC3-dependent pathway. It is interesting that C. immunis worsened DSS-induced colitis in C57BL/6 mice but protected against disease in Swiss Webster mice, a difference that may relate to differences in mouse strain or dose of DSS. Thus, it was demonstrated that C. immunis impacted two disparate phenotypes, with ILC3s serving as a common immunological node.

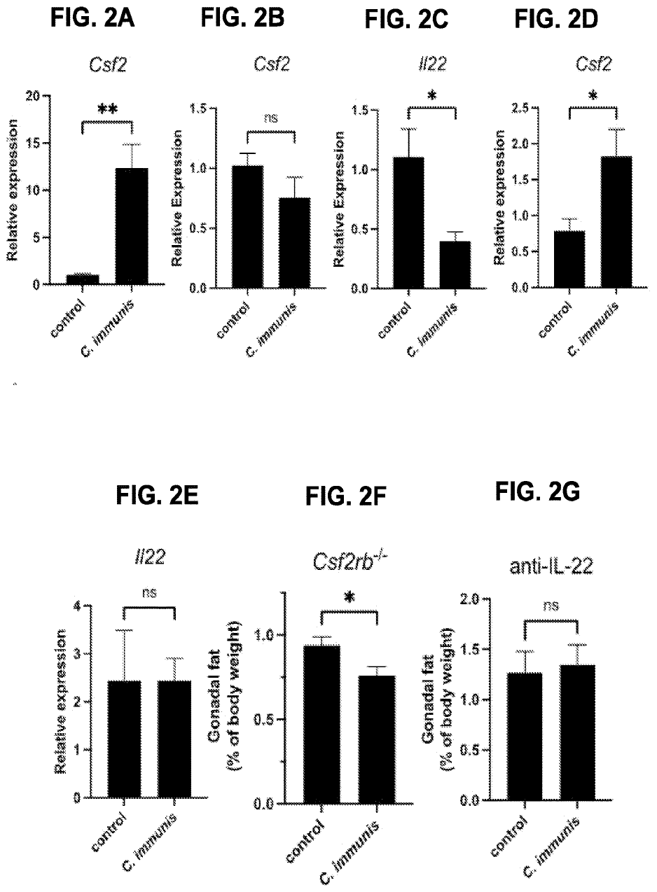

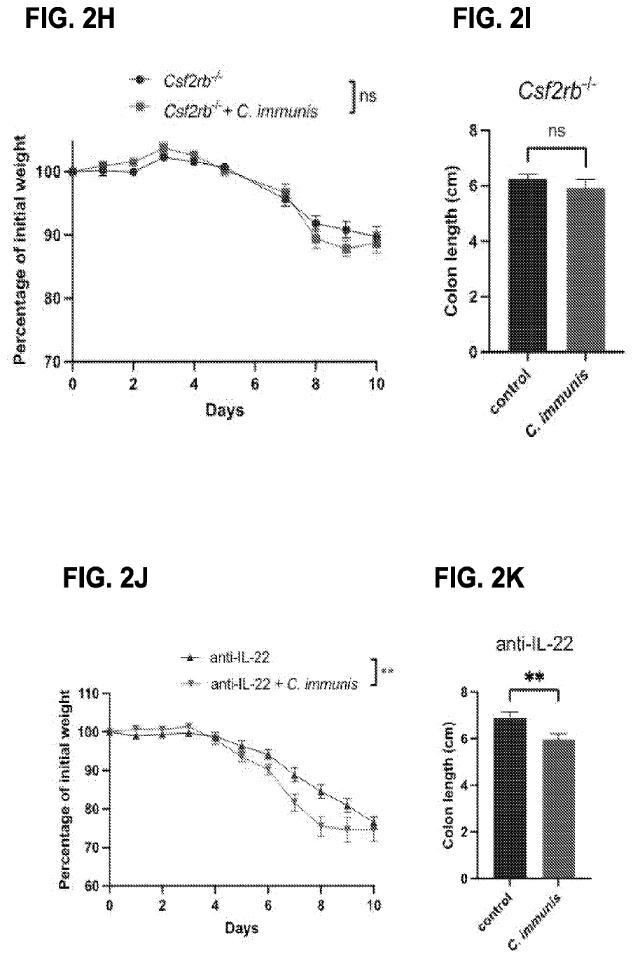

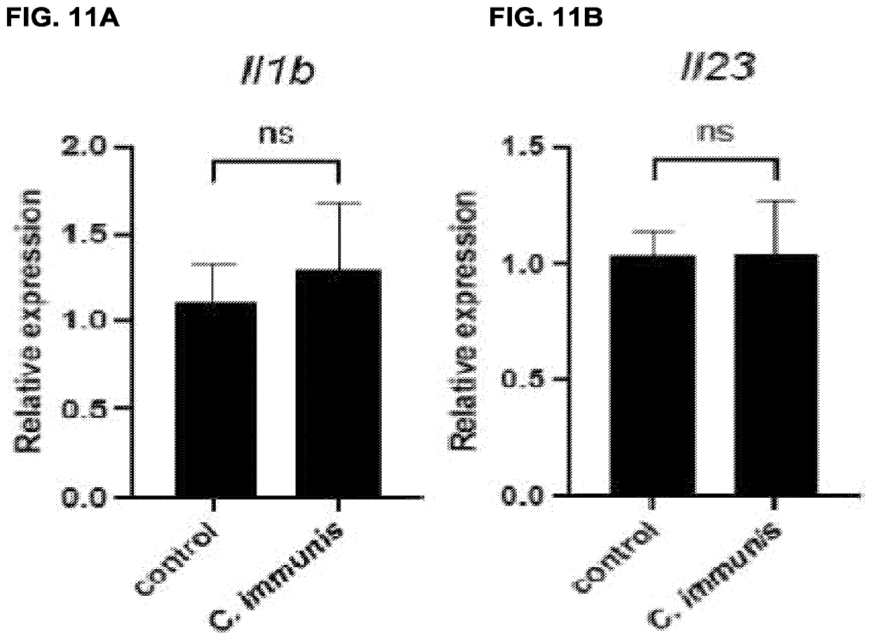

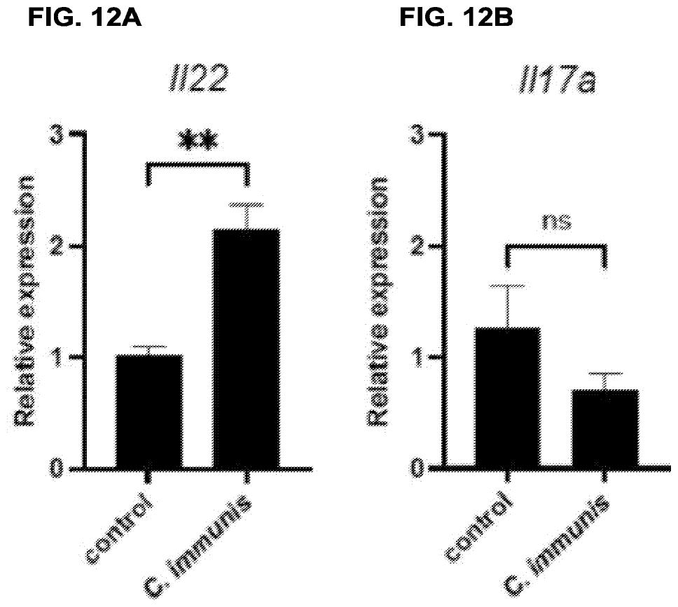

Although ILC3s are known to be regulated by the microbiota, there are limited examples of specific commensal bacteria that impact ILC3s directly. Given that numerous commensal bacteria affect myeloid cell secretion of cytokines (e.g., IL-23, IL-1b) that alter ILC3 function, it was asked whether C. immunis modulates ILC3s directly or influences their immunological milieu. It was found that expression of Il23 and Il1b was unchanged with C. immunis treatment (FIG. 11), a finding that indicates C. immunis was not altering immune signals upstream of ILC3s. Furthermore, treating MNK-3 cells, an ILC3-like cell line, with C. immunis led to robust induction of Il22 and Csf2, which encodes GM-CSF, with no impact on Il17a (FIG. 2A, FIG. 12). Collectively, these findings support the notion that C. immunis can directly modulate ILC3 function, with expression of specific cytokines impacted.

As ILC3s are major sources of intestinal IL-22 and GM-CSF, it was reasoned that treatment of mice with C. immunis leads to altered expression of these cytokines. In healthy SPF mice, C. immunis led to no change in Csf2 expression with altered levels of Il22 (FIG. 2B, FIG. 2C); however, in mice subjected to colitis, C. immunis increased expression of Csf2 with no effect on Il22 expression (FIG. 2D, FIG. 2E). Given this contextual regulation of ILC3-related cytokines, it was hypothesized they are differentially required for C. immunis-mediated modulation of adiposity and colitis. Indeed, the ability of C. immunis to reduce visceral fat was maintained in mice lacking CSF2RB, the receptor for GM-CSF, and lost when IL-22 was depleted (FIG. 2F, G). In contrast, C. immunis was able to modulate colitis severity in IL-22-depleted animals but not in Csf2rb/− mice (FIG. 2H-FIG. 2K). Taken together, the findings demonstrate that C. immunis differentially requires—and contextually regulates—IL-22 and GM-CSF for modulation of adiposity and colitis, respectively.

The Bacterial Determinant of C. immunis Activity:

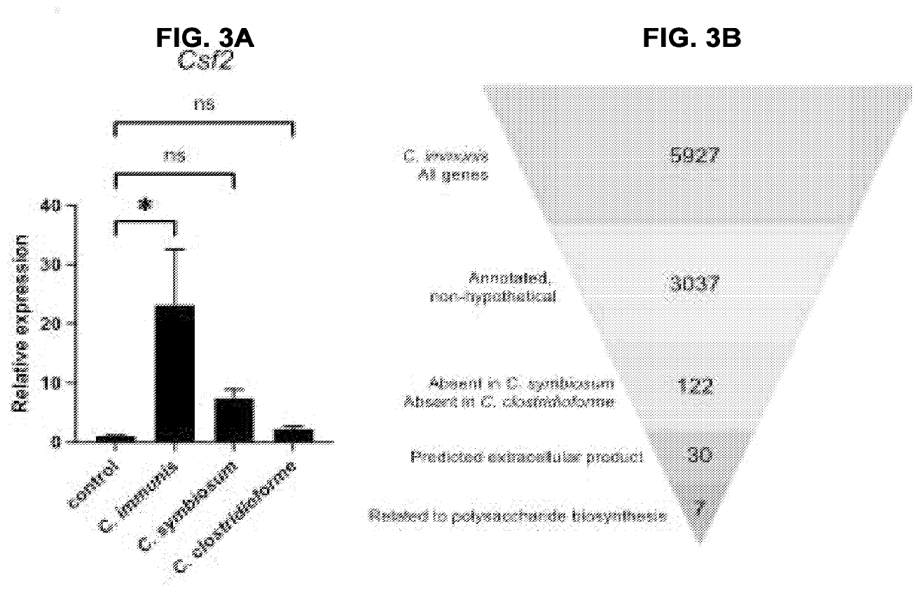

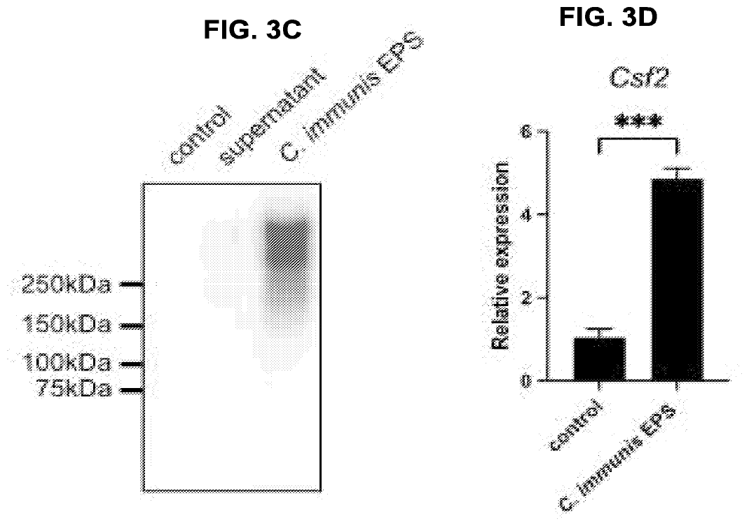

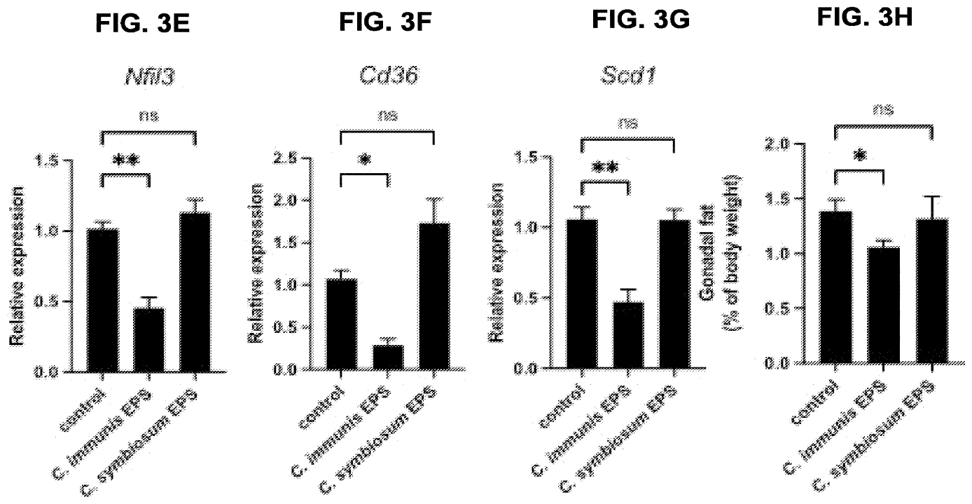

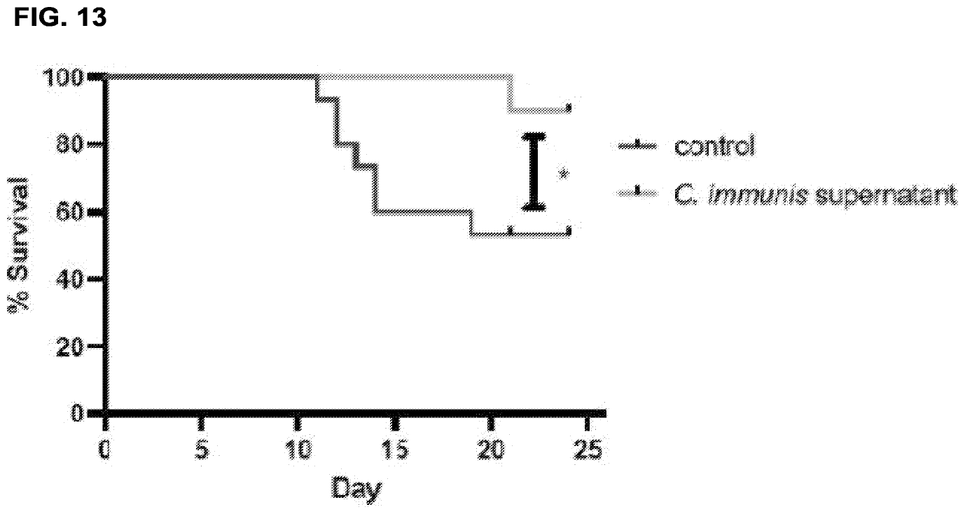

Having elucidated the key immunological determinants of C. immunis activity, it was sought to identify the bacterial factors required for modulating ILC3-mediated diseases. Given C. symbiosum and C. clostridioforme were unable to modulate adiposity (FIG. B), whether these organisms modulated ILC3 function was probed. Indeed, neither bacterium induced Csf2 expression in MNK-3 cells (FIG. 3A), which supported that C. immunis has some unique function lacking in these other closely related bacteria. With this in mind, the genomes for C. symbiosum and C. clostridioforme were sequenced and genomic comparisons with C. immunis were performed. Ultimately 122 genes were identified that were uniquely present in C. immunis (FIG. 3B). Based on the findings with MNK-3 cells, the bioactive molecule was present in the bacterial supernatant, which further confirmed that it was sufficient to modulate colitis outcomes (FIG. 13). As such, the list of 122 genes were manually curated to find those likely to produce extracellular products, which reduced the list to 30 genes. See Table 1 below.

TABLE 1
C. immunis genes associated with activity. Genes involved
in extracellular polysaccharide synthesis are listed in bold.

| Gene ID | Function |
|---|---|
| Ci_66 | Tripartite tricarboxylate transporter TctA family |
| Ci_131 | NAD-dependent oxidoreductase |
| Ci_271 | Protein-export membrane protein SecD/SecF |
| Ci_554 | Hydrolase of alpha/beta superfamily |
| Ci_{—}818 | capsular polysaccharide biosynthesis protein Cps4F |
| Ci_{—}820 | Capsular polysaccharide synthesis enzyme Cap5F |
| Ci_821 | UDP-N-acetyl-L-fucosamine synthase |
| Ci_891 | Pantothenate:Na+ symporter |
| Ci_1692 | Branched-chain amino acid transport system permease protein LivM |
| Ci_{—}2065 | capsular polysaccharide biosynthesis protein |
| Ci_{—}2145 | Pyrophosphorylase involved in lipopolysaccharide biosynthesis(LicC) |
| Ci_{—}2146 | Capsular polysaccharide biosynthesis protein(LicB) |
| Ci_2162 | Acyltransferase 3 |
| Ci_2655 | Oligopeptide ABC transporter, periplasmic oligopeptide-binding protein OppA |
| Ci_2783 | Multi antimicrobial extrusion protein (Na(+)/drug antiporter) |
| Ci_2978 | PTS system, mannitol-specific component |
| Ci_3330 | Glycosyltransferase, group 2 family |
| Ci_3460 | Cell surface protein |
| Ci_3691 | Glycoside-Pentoside-Hexuronide transporter |
| Ci_3700 | UDP-N-acetyl-L-fucosamine synthase |
| Ci_{—}3701 | Capsular polysaccharide synthesis enzyme Cap5F |
| Ci_3710 | Galacturonosyl transferase |
| Ci_3711 | Alpha-1,3-N-acetylgalactosamine transferase PgIA |
| Ci_3992 | Type IV fimbrial assembly protein PiIC |
| Ci_4441 | Sodium/glutamate symporter |
| Ci_4572 | Bacteriocin-like protein |
| Ci_5479 | Polygalacturonase |
| Ci_{—}5817 | Putative enzyme of poly-gamma-glutamate biosynthesis(capsule |
| | formation) |
| Ci_5925 | Choline binding protein A |
| Ci_5927 | Choline binding protein A |

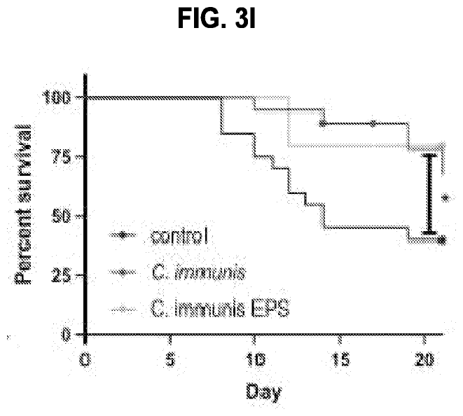

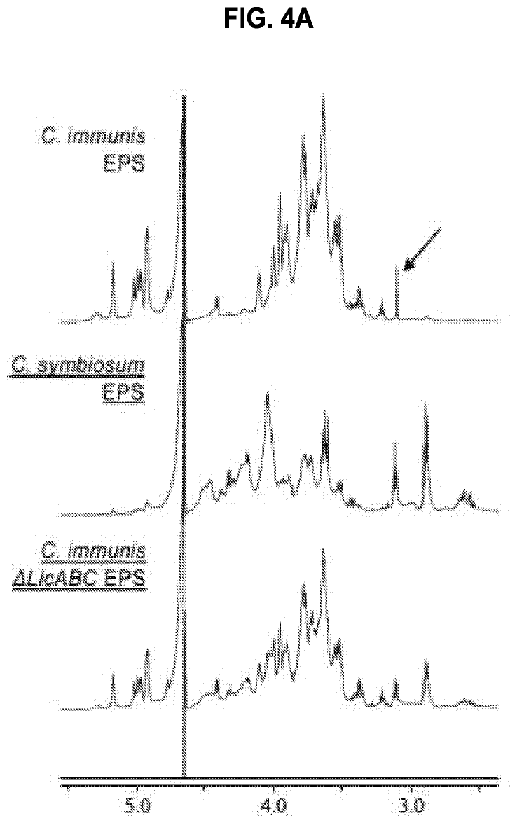

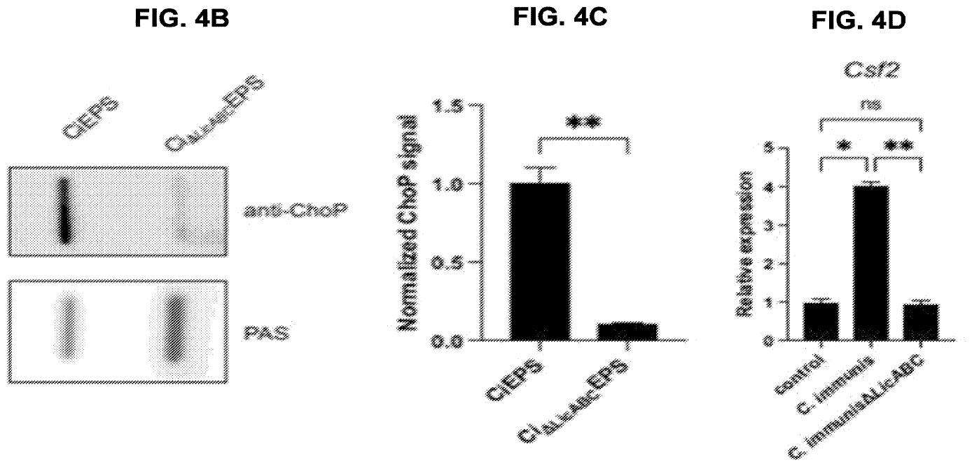

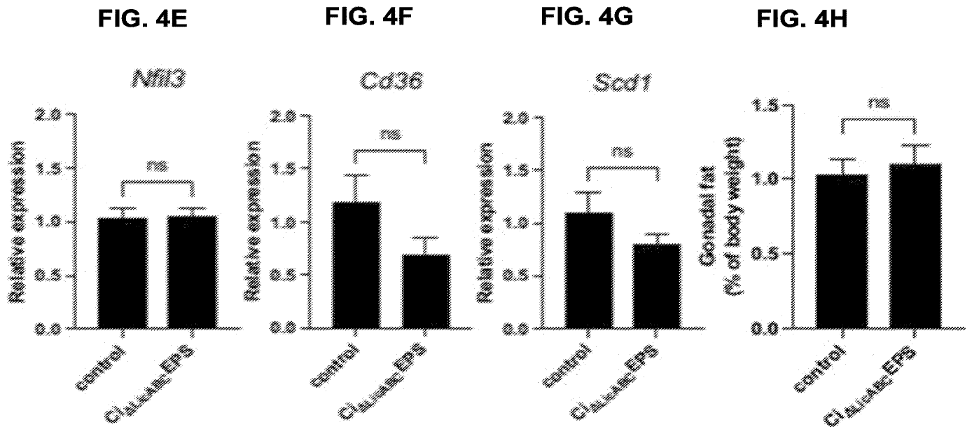

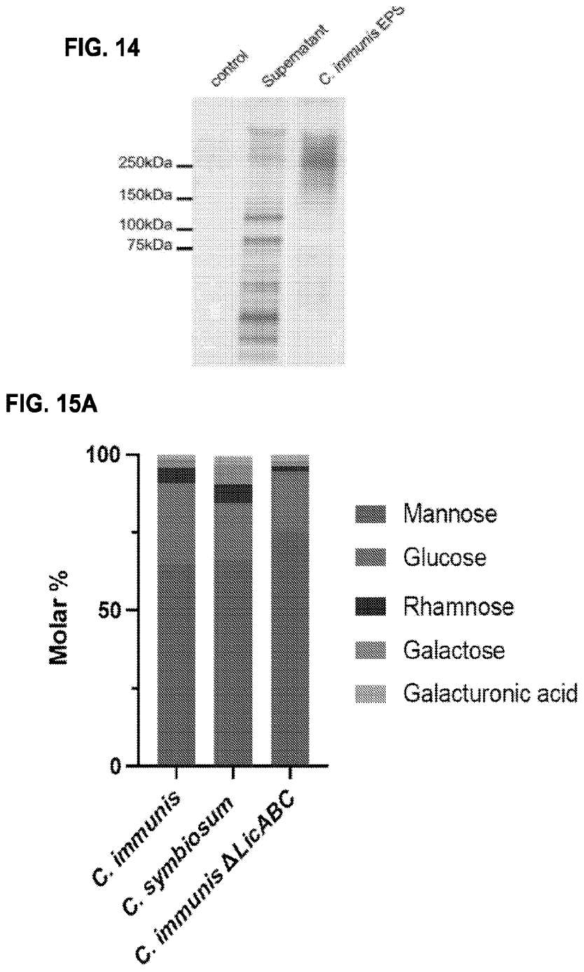

Intriguingly, nearly one-quarter of these genes were related to biosynthesis of an extracellular polysaccharide, a well-described mechanism by which commensal bacteria modulate the immune system. As such, supernatant high-molecular weight exopolysaccharides were purified from the C. immunis (EPS; FIG. 4C), which contained no appreciable protein contamination (FIG. 14). This purified EPS was sufficient to induce Csf2 expression in MNK-3 cells (FIG. 3D). Moreover, mice treated with EPS from C. immunis—but not EPS purified from C. symbiosum—had decreased small-intestinal expression of Nfil3, Cd36, and Scd1 as well as reduced levels of gonadal fat compared to control animals (FIG. 3E-FIG. 3H). Finally, C. immunis EPS phenocopied C. immunis in its ability to protect against colitis-associated death in Swiss Webster MMb mice (FIG. 3I). Taken together, the data established that this EPS was the relevant bioactive molecule from C. immunis. These data, which are consistent with the notion that ILC3s may directly sense bacterial ligands, extended the molecular mechanisms by which the microbiota regulates ILC3 function beyond a limited number of common bacterial metabolites.

Upon closer examination of the genes identified as distinguishing C. immunis from C. symbiosum and C. clostridioforme, two genes were chosen for additional experimentation, Ci2145 and Ci2146, that were homologous to a phosphocholine transferase (LicC) and a choline transporter (LicB), respectively. These genes reside in a three-gene operon that also contains a choline kinase gene (LicA; FIG. 16).

As known to the skilled person, LicA is phosphotransferase system (PTS) Lichenan-specific enzyme IIA component. A disclosed LicA can be identified by EnsemblBacteria CAB15883 and/or can be identified by Gene ID 937374. A disclosed LicA can comprise the nucleic acid sequence set forth in SEQ ID NO:25.

(SEQ ID NO: 25)
GTGAATGAGGAAATGGAACAAATCATTTTTCAAATCATCCTTCACGGAG
GAAACGGTAGGAGCTCCGCAATGGAGGCCATTGCCGCTGCGAAAAGCGG
TGACGCAGAAGAAGCGCGGAAAAAGCTTCAAGACGCTGCTGAAGAATTG
TCAAAAGCCCATCATTATCAGACAGAGCTGATCCAAAATGAAGCGGGCG
GAGAAAAGACGGAAATGACGCTTCTGATGGTTCATGCACAGGATCATTT
AATGAATGCAATGACAGTGAAGGATATGGCTGCAGAGATCATTGAGTTA
TATGAAAAAATCACCGAGCAAAGGGGAGCAAGCATATGA.

A disclosed LicA can be identified by UniProt P46319 and/or can be identified by Accession No. NP_391736.1. In an aspect, a disclosed licA can comprise the amino acid sequence set forth in SEQ ID NO:26.

(SEQ ID NO: 26)
MNEEMEQIIFQIILHGGNGRSSAMEAIAAAKSGDAEEARKKLQDAAEEL
SKAHHYQTELIQNEAGGEKTEMTLLMVHAQDHLMNAMTVKDMAAEIIEL
YEKITEQRGASI.

As known to the skilled person, LicB is phosphotransferase system (PTS) Lichenan-specific enzyme IIB component. A disclosed LicB can be identified by nsmblat n CAB15885 and/or can be identified by Gene ID 937379.

A disclosed licB can comprise the nucleic acid sequence set forth in SEQ ID NO:27.

(SEQ ID NO: 27)
ATGAATATATTACTCGTTTGTGCAGCAGGCATGTCTACTAGCTTACTGGTGAG
CAAGATGGAAAAAAGTGCGCAGGAACAGGGAAAAGACTATACCATTTGGGCAGTT
TCAGGCGATTCTGTACAAAATCATATCGATAAAGCGGATGTATTGCTTCTCGGTCCG
CAAGTCCGTTACATGCTTCCGCAGCTCAAAAAATTAGGAGAGTCAAAGGGGGTTCC
GGTAGACGTCATTAATACCGTCCATTATGGAACGTGCAATGGGGCGGAAGTGCTGA
AATCAGCTGAACAGCTTGGACATGTGTCATAG.

A disclosed LicB can be identified by UniProt P46318 and/or can be identified by Accession No. NP_391738.1. A disclosed LicB can comprise the amino acid sequence set forth in SEQ ID NO:28.

(SEQ ID NO: 28)
MNILLVCAAGMSTSLLVSKMEKSAQEQGKDYTIWAVSGDSVQNHIDKAD
VLLLGPQVRYMLPQLKKLGESKGVPVDVINTVHYGTCNGAEVLKSAEQL
GHVS.

As known to skilled person, LicC is phosphotransferase system (PTS) Lichenan-specific enzyme IIB component. A disclosed LicC can be identified by Ensembl CAB15884 and/or can be identified by Gene ID 937375. In an aspect, a disclosed LicC can comprise the nucleic acid sequence set forth in SEQ ID NO:29.

(SEQ ID NO: 29)
GTGAATAAAGTCAATCAGATCTTAGAAGAAAAGGTCATGCCTATAGCGGGA
AGAATTGCGGGACAGCGTCATTTGCAGGCGCTGCGTGATGGTATCATCCTCACGAT
GCCCCTGATTATCATCGGTTCCTTTTTCCTCATCATTGGCAACCTCCCGATCCCGGGA
TACGCTGAGTTTATGGCAAAAACGTTTGGCAGCTCCTGGTCTGAAAAACTGGCCTAC
CCAGTCGACGCGACCTTTGAAATTATGGGTCTTGTCGCCGCGTTCGGCATTGCCTAC
CGGCTCGCCGAGAAATACGGTGTTGATGCCTTGTCGGCCGGAGCCATTTCCCTTGCG
GCATTTTTATTAGCAACACCTTATCAAGTGCCGTTTATGCCGGATGGCGCGACGAAA
GAAATCATGGTTGGCGGCGGGATTCCGCTTTCGCTCATGGGAAGCAAAGGCTTATTT
GTCGCCATGATTATCGCAATGGTGTCCACAGAGATTTACCGCCTTATTATCCAGCGC
AATTTAGTCTTCAAAATGCCGGACGGTGTCCCGCCGGCGGTAAGCAAGTCATTTGTC
GCGTTAATCCCCGGCTTTGCCGTCATCTTCCTAATTTGGGCAGCCCGGCTGATTGTT
GAAGCGACACCGTTTGAAAGCCTTCATAATATTGTAAGCGTTTTATTGGGGACTCCG
CTTTCCATTCTGGGAGGAAGCCTTGGAGGAAGCCTTGTCGCCGAAGCCGTCAAAAT
GCTTTTATGGGCCTGCGGCCTTCACGGTGCAAACATTGTTGGCGGCGTCATGGCACC
GATTTGGTACGGCGCAATGGATGCCAACCGGATCGCATTCCAAGCCGGTGAGGAGC
TTCCTAAGATTTTCACCCAGCAATTTTTCGATATCTGGGTCAATATTGGCGGAAGCG
GCGCCACATTGGCACTGGTTGTCACGATGTTCCTGCGCGCCCGAAGCAAACAAATG
AAGCAGCTCGGGAAGCTTGCGGTCGGCCCGGCCATCTTCAATATTAACGAACCGAT
TATATTCGGAATGCCGATCGTCATGAACCCAATGCTCTTACTTCCGTTTATCATCAC
GCCGCTTGTCACAGTGACACTCACGTACATCGGAATGAGCACCGGCCTTGTCGCCA
AACCGGCGGGTATCGCAGTGCCGTGGACCATGCCGCCGATCTTTTCAGGCTACTTGG
CGACAGGAGGAAAAGTCTCAGGCGCGGTCATGCAAGCCATTAATATTGCCGTGTCC
TTTGTTGTCTACTATCCATTCTTCAGAATGTGGGATAAGCAGAAGCTGAAAGAAGA
AAACGATCTGGAGCTGGTCCAAACGCCTGCAGCAACAGATGACAAAGAAGCGGCT
CTGTAG.

A disclosed LicC can be identified by UniProt P46317 and/or can be identified by Accession No. NP_391737.1. A disclosed licC can comprise the amino acid sequence set forth in SEQ ID NO:30.

(SEQ ID NO: 30)
MNKVNQILEEKVMPIAGRIAGQRHLQALRDGIILTMPLIIIGSFFLIIGNLPIPGYAE
FMAKTFGSSWSEKLAYPVDATFEIMGLVAAFGIAYRLAEKYGVDALSAGAISLAAFLLA
TPYQVPFMPDGATKEIMVGGGIPLSLMGSKGLFVAMIIAMVSTEIYRLIIQRNLVFKMPD
GVPPAVSKSFVALIPGFAVIFLIWAARLIVEATPFESLHNIVSVLLGTPLSILGGSLGGSLV
AEAVKMLLWACGLHGANIVGGVMAPIWYGAMDANRIAFQAGEELPKIFTQQFFDIWV
NIGGSGATLALVVTMFLRARSKQMKQLGKLAVGPAIFNINEPIIFGMPIVMNPMLLLPFII
TPLVTVTLTYIGMSTGLVAKPAGIAVPWTMPPIFSGYLATGGKVSGAVMQAINIAVSFV
VYYPFFRMWDKQKLKEENDLELVQTPAATDDKEAAL.

ABC operon is required for activity, a system to genetically manipulate C. immunis was developed. Although CRISPR-Cas9 has been a useful approach to make isogenic mutants in some clostridial species, expression of Cas9 was toxic to C. immunis, similar to its effect in many other commensal bacteria. Instead, a suicide plasmid that contained an erythromycin resistance cassette flanked by DNA homologous to C. immunis LicA was created to facilitate homologous recombination that disrupted the entire LicABC locus (FIG. 16B). EPS isolated from the C. immunis ΔLicABC isogenic mutant (CiΔLicABCEPS) still had the same monosaccharide composition as that from wild-type C. immunis (FIG. 15), but it lacked phosphocholine as assessed by ¹H-NMR and immunoblot (FIG. 4A-FIG. 4C). Thus, 3 orthogonal methods—¹H-NMR, bacterial genetics, and immunoblotting—have been used to demonstrate C. immunis EPS contains phosphocholine. Consistent with phosphocholine being critical for the activity of C. immunis EPS, supernatant from C. immunis ΔLicABC was unable to induce Csf2 expression in MNK-3 cells (FIG. 4D). Moreover, mice treated with CiΔLicABCEPS had small-intestinal expression of Nfil3, Cd36, or Scd1 and levels of adiposity that were indistinguishable from control animals (FIG. 4E-FIG. 4H). Collectively, these data clearly demonstrated that the phosphocholine present on C. immunis EPS facilitated its function.

Through a combination of computational, genetic, and biochemical approaches, molecular Koch's postulates have conceptually fulfilled for the role of the C. immunis LicABC locus in modulating ILC3-regulated diseases. As the ability to culture and genetically manipulate commensal organisms continues to advance, demonstrating the genetic and structural basis for how the microbiota impacts host physiology will become more commonplace, just as it has become de rigueur in microbial pathogenesis.

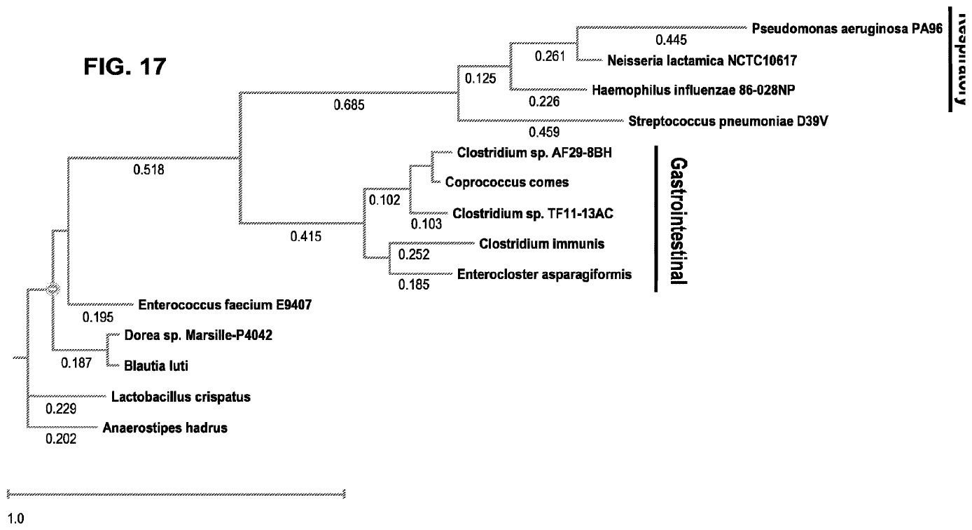

The addition of phosphocholine as a structural modification can be a common strategy employed by intestinal commensal bacteria in modulating host function, similar to its described role in respiratory pathogens. Indeed, several taxonomically diverse commensal bacteria have been identified that contain a LicABC locus homologous to that of C. immunis (FIG. 17). Intriguingly, the gene sequences for bacteria primarily found in the respiratory tract cluster separately from intestinal organisms, which itself forms two distinct clades (FIG. 17). Given the phylogeny of the gene sequences is distinct from the bacterial taxonomic relationships, it is possible the LicABC locus is spread via horizontal gene transfer. Along these lines, it is interesting to note that the C. immunis LicABC operon is flanked by mobile genetic elements (FIG. 16A), which may help explain why closely related taxa lacked these genes. It is tempting to speculate that modification of common microbe-associated molecular patterns with phosphocholine and other similar small moieties may act to fine-tune the host response, thus providing increased specificity in how commensal bacteria regulate host responses.

iii. Materials and Methods

### Mice:

Ahrfl/fl (stock #006203)51, Rorc-cre (stock #022791)25, and Rag1−/− mice (stock #002216)52 were obtained from The Jackson Laboratory. Csf2rb−/− mice were obtained from Mari Shinohara (Duke University). Gnotobiotic Swiss Webster MMb mice were obtained from Dennis Kasper (Harvard University); these mice were co-housed with germ-free C57BL/6 mice to generate C57BL/6 MMb mice. All animals were bred and maintained in the animal facility at Duke University, and the gnotobiotic mice were bred and maintained in vinyl isolators in the Duke Gnotobiotic Core. Experimental manipulation of gnotobiotic mice was performed in autoclaved, individually ventilated cages in which animals received autoclaved food (LabDiet 5K67) and water. Mice used in experiments were sex- and age-matched and drawn randomly from the same litter, when feasible. All procedures were approved by Duke's Institutional Animal Care and Use Committee and were conducted in accordance with National Institutes of Health guidelines.

### Bacteria:

C. immunis was obtained from Dennis Kasper (Harvard University). C. immunis, C. symbiosum (DSM 29356), and C. clostridioforme (DSM 933) were grown in peptone yeast glucose (PYG) broth (Anaerobe Systems) or on brain heart infusion-supplemented (BHI-S) agar plates (ATCC Medium 1293). All clostridial strains were grown in an anaerobic chamber (Coy laboratories) with 2.5% H₂ and 0 ppm O₂ at 37° C. Escherichia coli S17λPir was grown aerobically in LB at 37° C.

### RNA-Sequencing and Analysis:

One week after orally administering C. immunis (200 μL containing ~108 colony-forming units [CFU]) or, as a control, sterile PYG media (200 μL) to male C57BL/6 MMb mice (12 weeks of age), the proximal 2 cm of colon was collected, frozen immediately in liquid nitrogen, and stored at −80° C. until needed. Tissues were homogenized in Trizol (Invitrogen) using a bead-beating approach, and RNA was purified according to the manufacturer's instructions. RNA Integrity Number (RIN) scores for all samples used for RNA-seq were >7.0 as assessed on a TapeStation 2200 (Agilent). cDNA libraries were generated using a mRNA HyperPrep kit (KAPA) and sequenced on aNovaSeq6000 (Illumina; S4 flow cell with 150 bp paired-end reads).

FastQC (version 0.11.9) was used to assess read quality and to perform trimming, STAR (release 2.7.9a) was used to align reads to the mouse genome mm10 (GRCm39), and FeatureCounts was used to generate a count table. Differential gene analysis was performed with DESeq2 (version 1.28.1), and a volcano plot was generated with EnhancedVolcano. A preranked gene set enrichment analysis was performed using GSEA 4.3.2 using a previously described NFIL3-dependent gene set as input. R (version 4.0.0) was used for all analyses.

Real-Time qPCR Analysis:

RNA was isolated from tissue as described above. For cells, Trizol was added to samples, and RNA was purified according to the manufacturer's instructions. cDNA was prepared using random hexamers and the High-Capacity cDNA synthesis kit (Applied Biosystems), and qPCR was performed on a Step One Real Time PCR system (Applied Biosystems) with iTaq Universal SYBR Green Supermix (Bio-Rad).

The primers are summarized in Table 2 provide below.

TABLE 2
Primers for Real-time qPCR Analysis

| Description | Sequence | SEQ ID NO. |
|---|---|---|
| Gadph fwd | ACCACAGTCCATGCCATCAC | SEQ ID NO: 1 |
| Gadph rev | TCCACCACCCTGTTGCTGTA | SEQ ID NO: 2 |
| Nfil3 fwd | CTTTCAGGACTACCAGACATCC | SEQ ID NO: 3 |
| | AA | |
| Nfil3 rev | GATGCAACTTCCGGCTACCA | SEQ ID NO: 4 |
| Cd36 fwd | TCATATTGTGCTTGCAAATCCA | SEQ ID NO: 5 |
| | A | |
| Cd36 rev | TGTAGATCGGCTTTACCAAAGA | SEQ ID NO: 6 |
| | TG | |
| Scd1 fwd | CTTCTTCTCTCACGTGGGTTG | SEQ ID NO: 7 |
| Scd1 rev | CGGGCTTGTAGTACCTCCTC | SEQ ID NO: 8 |
| Il23 fwd | CATGCTAGCCTGGAACGCACAT | SEQ ID NO: 9 |
| Il23 rev | ACTGGCTGTTGTCCTTGAGTCC | SEQ ID NO: 10 |
| Il1b fwd | TGGACCTTCCAGGATGAGGACA | SEQ ID NO: 11 |
| Il1b rev | GTTCATCTCGGAGCCTGTAGTG | SEQ ID NO: 12 |
| Il22 fwd | GCTTGAGGTGTCCAACTTCCAG | SEQ ID NO: 13 |
| Il22 rev | ACTCCTCGGAACAGTTTCTCCC | SEQ ID NO: 14 |
| Csf2 fwd | AACCTCCTGGATGACATGCCTG | SEQ ID NO: 15 |
| Csf2 rev | AAATTGCCCCGTAGACCCTGCT | SEQ ID NO: 16 |
| Il17a fwd | ATCCCTCAAAGCTCAGCGTGTC | SEQ ID NO: 17 |
| Il17a rev | GGGTCTTCATTGCGGTGGAGAG | SEQ ID NO: 18 |

The comparative Ct method was used to quantify transcripts that were normalized with respect to Gapdh.

### Assessment of Adiposity:

Mice (13-18 weeks of age) were orally administered either ~108 CFU of bacteria, 4 mg of exopolysaccharide, or sterile PYG media. One week later, the gonadal fat pads were collected. The fat pads surrounding the ovaries and fallopian tubes were collected in female mice, while the epididymal fat pads were collected in male mice. The weight of these gonadal fat pads was normalized to the animal's body weight at time of sacrifice.

### Dextran Sodium Sulfate (DSS) Colitis:

DSS experiments were performed as previously described. Mice were orally administered C. immunis (200 μL; ~108 CFU), clarified C. immunis supernatant (200 μL), or EPS as described below. For initiation of colitis, the mice were given DSS ad libitum in their drinking water for 7 days, with the DSS solution changed every 2-3 days. From day 7 through day 10, the mice were given water without DSS. C57BL/6 mice were orally administered C. immunis or EPS (4 mg) on days 7 and 0, with day 0 representing initiation of 2.5% DSS (molecular mass 36-50 kDa; Affymetrix). Swiss Webster MMb mice were orally administered C. immunis, clarified C. immunis supernatant, or EPS (2 mg) three times per week starting on day −7 and continuing throughout the entire experiment. Consistent with previous work, Swiss Webster MMb mice received 4% DSS (molecular weight 36-50 kDa; MP Biomedical). Animals were weighed every 1-2 days, and any mouse that appeared moribund was euthanized. Ten days after initiation of DSS, colons were harvested for qPCR and histological analyses. Two independent investigators conducted a histological assessment of colons, with scores representing the combination of epithelial damage, mucosal inflammation, and extent of disease; all elements were scored 0-3, with 0 being normal.

### Cell Culture:

All tissues and cells were cultured in a humidified incubator with 5% CO₂ at 37° C. MNK-3 cells were obtained from Maria Ciofani (Duke University) and maintained as previously described, adding recombinant mouse IL-7 (10 ng/mL; BioLegend) and IL-15 (10 ng/mL; BioLegend) every 3 days. Cells (5×10⁵) were plated in 24-well plates. The following day, cells were treated with bacterial supernatants (10% v/v) or EPS (0.5 mg/ml) for 4 hours and lysed for RNA isolation. For experiments with MNK-3 cells, bacterial supernatants were prepared by growing bacteria anaerobically in PYG overnight at 37° C., resuspending the bacteria in an equal volume of peptone water (10 g/L peptone, 5 g/L sodium chloride), incubating anaerobically overnight at 37° C., and collecting the supernatants after centrifugation.

### IL-22 Depletion:

Mice were injected intraperitoneally with either a neutralizing antibody against IL-22 (150 μg/dose; clone 8E11; Genentech) or an IgG1 isotype control (150 μg/dose; Genentech) every other day. For experiments assessing gonadal fat, the antibody dosing regimen began 2 days prior to treatment with C. immunis. For DSS colitis experiments, mice were treated with the antibodies beginning 2 days before the initiation of DSS.

### Genomic Comparisons of Clostridium Species:

Genomic DNA was isolated from overnight cultures of C. immunis, C. symbiosum, and C. clostridioforme using a MagAttract HMW DNA kit (Qiagen). A multiplexed sequencing library was generated using a SMRTbell prep kit (PacBio) and sequenced on a PacBio RS. Flye (release 2.8.3) was used to perform de novo assembly on quality-filtered reads, which resulted in a complete and circularized genome for C. immunis (genome size of 5.34 Mbp), 108 contigs (genome size of 5.54 Mbp) for C. symbiosum, and 18 contigs (genome size of 5.84 Mbp) for C. clostridioforme. Gene annotation was performed with RAST61, and a genome-wide, sequence-based comparison of the three strains was performed using The SEED Viewer web server (version 2.0). Genes that were uniquely present in C. immunis were further manually curated to identify genes that are likely to generate extracellular products.

Generation of C. immunis ΔLicABC:

It was established that pMTL82151, one of the ClosTron plasmids, fails to replicate in C. immunis and therefore could be used as a suicide vector. The Q5 High-Fidelity DNA polymerase (NEB) was used to PCR amplify the ErmB gene that provides resistance to erythromycin (using pMTL83251 as the template) and two 800 bp regions (separated by 80 bp) that flanked the LicA target site.

The PCR primers are summarized in Table 3 provide below.

TABLE 3
Primers for PCR

| Description | Sequence | SEQ ID NO. |
|---|---|---|
| LicA Left F | gctcggtacccggggatcctAATACCGTCAGCTACACTG | SEQ ID NO: 19 |
| LicA Left R | cttcggccggTAGGCCGGACATATTCTATATC | SEQ ID NO: 20 |
| LicA Right F | gaatgtgtttTTTCTATATGGTGATCTTAGTAAAC | SEQ ID NO: 21 |
| LicA Right R | agcttgcatgtctgcaggccTTTAATTGGACAAAGCGTC | SEQ ID NO: 22 |
| ErmB F | gtccggcctaCCGGCCGAAGCAAACTTAAG | SEQ ID NO: 23 |
| ErmB R | catatagaaaAAACACATTCCCTTTAGTAACGTG | SEQ ID NO: 24 |

After digesting these three PCR products with XbaI and XhoI, they were cloned into an XbaI- and XhoI-digested pMTL82151 plasmid using HiFi assembly (NEB). Electrocompetent E. coli S17λPir was transformed with the ligation mixture, which generated S17λPir:pMTL82151ΔLicABC. This strain was used to transfer pMTL82151ΔLicABC into C. immunis using standard conjugation methods. Overnight cultures of E. coli donors and C. immunis recipients were mixed at a 10:1 donor-to-recipient ratio. Multiple aliquots (30 μL) of the mixture were spotted onto a BHI-S agar plate and incubated anaerobically at 37° C. After 24-48 h, the total growth on each plate was collected, resuspended in fresh media, and grown on BHI-S plates containing erythromycin (500 μg/mL) and colistin (10 μg/mL). To confirm that the plasmid was not present in C. immunis, the culture was also grown on BHI-S plates containing colistin (10 μg/mL) and chloramphenicol (12.5 μg/mL), against which the plasmid backbone confers resistance. Erythromycin-resistant colonies of C. immunis were confirmed to harbor the insertion of the ErmB gene and expected 80-bp deletion in LicA by PCR and Sanger sequencing.

### EPS Purification:

The supernatant from overnight bacterial cultures was concentrated ~50-fold using a 100 kDa molecular weight cutoff (MWCO) spin column (Amicon Ultra, Millipore). The retentate was incubated overnight with DNase I (50 μg/mL) and Rnase A (50 μg/ml) at 37° C. followed by an overnight incubation with proteinase K (500 μg/mL) at 37° C. The volume of the resultant mixture was increased to 15 mL with dH₂O and concentrated back down to 1-2 mL using a 100 kDa MWCO column to remove proteinase K. The retentate was precipitated with ice-cold ethanol (80% final volume) at −20° C. overnight. Precipitates were air-dried and resuspended in distilled water.

### Visualization of EPS by SDS-PAGE:

EPS (60 μg) was boiled for 5 mins in Laemmli buffer with β-mercaptoethanol and run on a 10% polyacrylamide gel (MiniPROTEAN TGX, BioRad) at 200 V for 1 hr. Gels were visualized with either a silver stain (Thermo Fisher Pierce) following the manufacturer's instructions or subjected to periodic acid-Schiff (PAS) staining. For the latter, the gel was washed in deionized water for 10 min, fixed in 12.5% trichloroacetic acid for 30 min, oxidized with 1% periodic acid for 1 hr., washed in deionized water for 4-5 hr, and stained with Schiff's reagent for 1 hr. After staining, the gel was reduced with three 10 min washes in 0.5% sodium metabisulfite.

### Slot Immunoblot:

EPS (5 μg) was added to a methanol-activated PVDF membrane housed in a slot blot manifold (Hoefer Inc.). The membrane was blocked in 3% bovine serum albumin in TBS containing 0.1% Tween 20 (TBS-T) for 1 hr., followed by overnight incubation at 4° C. with an anti-phosphocholine antibody (1:500 dilution; clone BH8; Millipore). After washing with TBS-T, the membrane was incubated with an HRP-conjugated secondary antibody (Thermo Fisher Scientific) for 1 hr. Chemiluminescence generated by treatment with Clarity ECL (Bio-Rad) was imaged on an Odyssey XF system (LI-COR Biosciences). EPS-treated membranes were also stained with PAS as detailed above to visualize the amount of carbohydrate loaded.

### Glycosyl Composition Analysis by GC-MS of TMS-Derivatized Methyl Glycosides:

Glycosyl composition analysis was performed by combined gas chromatography-mass spectrometry (GC-MS) of the per-O-trimethylsilyl (TMS) derivatives of the monosaccharide methyl glycosides produced from the sample by acidic methanolysis as described previously. Briefly, samples (150-300 μg) were heated with 1 M methanolic HCl in a sealed screw-top glass test tube for 18 hr. at 80° C. After cooling and removal of the solvent under a stream of nitrogen, the samples were re-N-acetylated and dried again. The samples were then derivatized with Tri-Sil® (Pierce) at 80° C. for 30 min. GC-MS analysis of the TMS methyl glycosides was performed on an Agilent 7890A GC interfaced to a 5975C MSD, using a Supelco Equity-1 fused silica capillary column (30 m×0.25 mm ID).

### NMR Spectroscopy:

Each lyophilized EPS sample (~1-2 mg) was dissolved in 500 μl of D₂O (99.9% D, Sigma) and placed in a 5-mm NMR tube. Sodium trimethylsilylpropanesulfonate (0.5 μL) was added as a reference. Liquid ¹H-NMR data were obtained at 298 K on a Varian VNMRS spectrometer (TH, 599.66 MHz). ¹H-NMR parameters: 2.0 s relaxation delay, 65536 Hz spectral width, 16384 data points and 64 transients with total recycle delay of 3.3 s between each transient. The experiment was performed with suppression of the HOD signal at 4.78 ppm by presaturation. Prior to the Fourier transformation, the data were apodized with an exponential decay function with line broadening of 0.5 Hz, 900 sine square, and zero-filled to 64 k points. The baselines were corrected automatically by subtracting a 3rd-order Bernstein polynomial fit. The spectra were processed and analyzed with MestreNova (version 14.2.1-27684).

Phylogeny of C. immunis LicABC Homologs:

Homologs of the C. immunis LicABC locus were identified using BLASTn (2.13.0). The gene sequences for these LicABC loci as well as the sequences from select organisms primarily found in the respiratory tract were aligned using ClustalW. A maximum likelihood phylogenetic tree was generated using RAxML as implemented with the default settings in MegAlign Pro (DNA Star).

### Statistics:

Sample-size estimates for each experiment were based on previous laboratory experience. The investigators were not blinded to allocation during experiments and outcome assessment. Prism 9 (GraphPad Software) was used for all statistical analyses unless otherwise specified.

### B. Example 2 Data

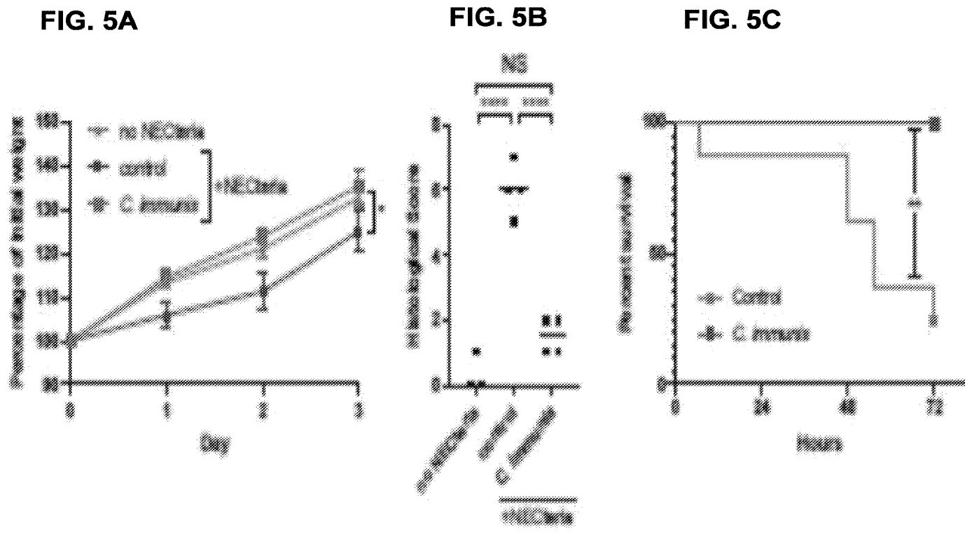

The inventors determined that C. immunis protects against disease in two different mouse models of necrotizing enterocolitis (NEC; FIG. 5), a devastating disease of newborns that disproportionately affects premature infants and has a mortality rate of ~30%. Data from mouse models indicates that ILC3s are pathogenic in NEC, which likely explains the protective effect of C. immunis.

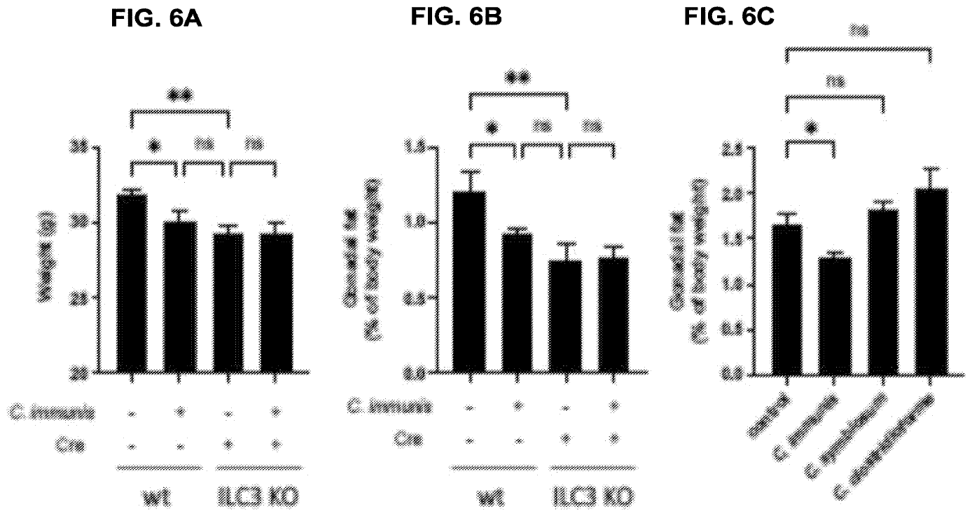

Additionally, transcriptional profiling revealed that C. immunis inhibited expression of many metabolism-related genes. Given that this gene signature is involved in ILC3-mediated control of obesity, it was reasoned that C. immunis may impact weight and fat in mice. Indeed, mice treated with C. immunis had lower body weights and less visceral fat (assessed as gonadal fat), changes that were ILC3-dependent (FIGS. 6A, 6B). Importantly, mice treated with C. symbiosum or C. clostridioforme, the two bacterial species that are genetically most identical to C. immunis, had no change in body fat (FIG. 6C). These findings indicate that this effect of C. immunis is relatively unique among other closely related bacteria.

Identification of the C. immunis Bioactive Product:

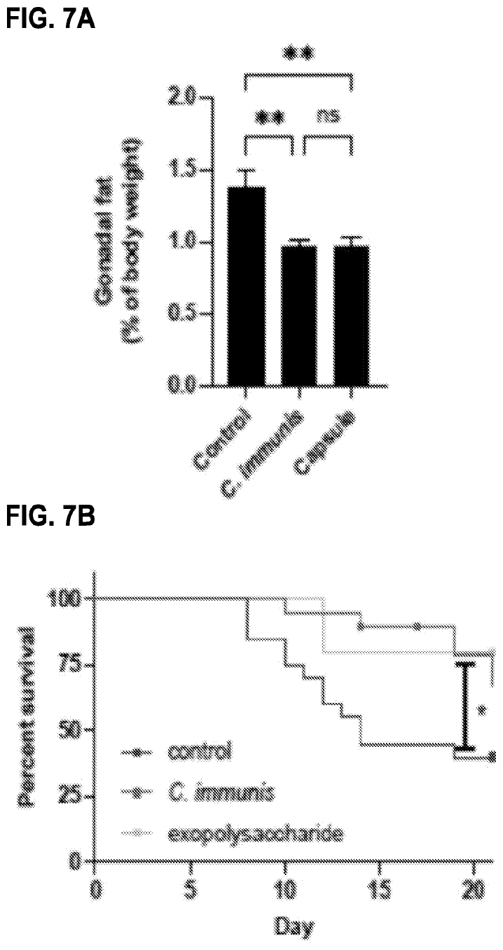

Knowing that the activity of C. immunis was distinct from that of C. symbiosum and C. clostridioforme, genomic comparisons were performed between these 3 strains and identified 41 genes that were present in C. immunis but absent in the other 2 strains. Given that the active product was present in the supernatant, this list was manually curated for genes that would result in extracellular and/or secreted products, which narrowed the list to 8 genes. Intriguingly, one of the genes related to biosynthesis of a polysaccharide capsule, a previously described mechanism by which some other bacteria modulate the immune system. Inspection of the C. immunis genome revealed a locus with several genes relevant to capsule production, and electron microscopy was indicative of a capsule being present. Accordingly, high molecular weight polysaccharides were purified from C. immunis supernatant by treating it with protease, DNase, and RNase, filtering through a 50 kDa cutoff filter, and ethanol precipitating the remaining polysaccharides. Mice treated with this purified, high-molecular weight exopolysaccharide had visceral fat amounts indistinguishable from C. immunis-treated mice and lower than untreated mice (FIG. 7A), a finding that demonstrates that this exopolysaccharide was sufficient to modulate visceral fat. Additionally, mice treated with the C. immunis exopolysaccharide had decreased colitis severity that phenocopied C. immunis-treated mice (FIG. 7B). Taken together, the findings establish that the purified C. immunis exopolysaccharide is sufficient to modulate both obesity and inflammatory phenotypes.

### C. Example 3—Immune Checkpoint Inhibition as it Relates to Cancer Therapy

C. immunis Enhanced the Efficacy of Anti-PD1 Therapy in Cancer:

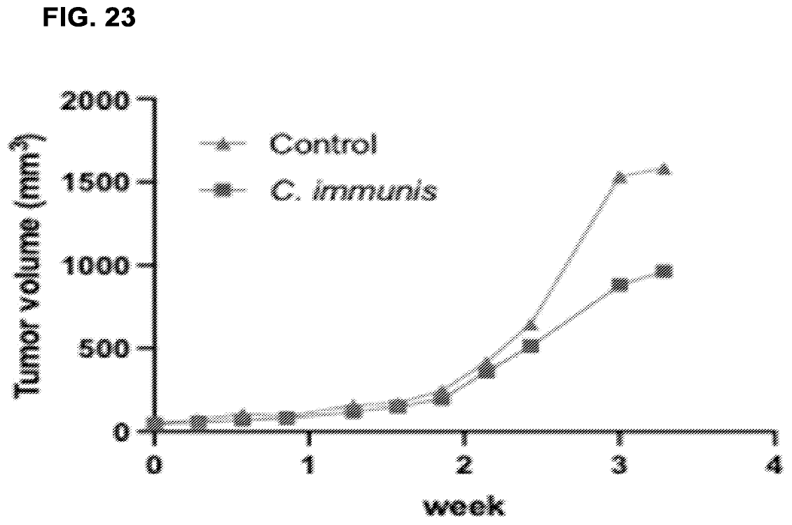

Referring to FIG. 23, MC38 cells were implanted subcutaneously in the flanks of mice. When tumors were 50-100 mm³ in size, mice began receiving injections with anti-PD1 every 3 days×4-5 doses. One group of mice received oral treatments with C. immunis 3×/week, starting one week before tumor implantation and continuing for the duration of the experiment. As can be seen on the graph, by week 3 tumor growth had reduced compared with Control group.

### D. Example 4—Necrotizing Enterocolitis

C. immunis Protects Against Necrotizing Enterocolitis:

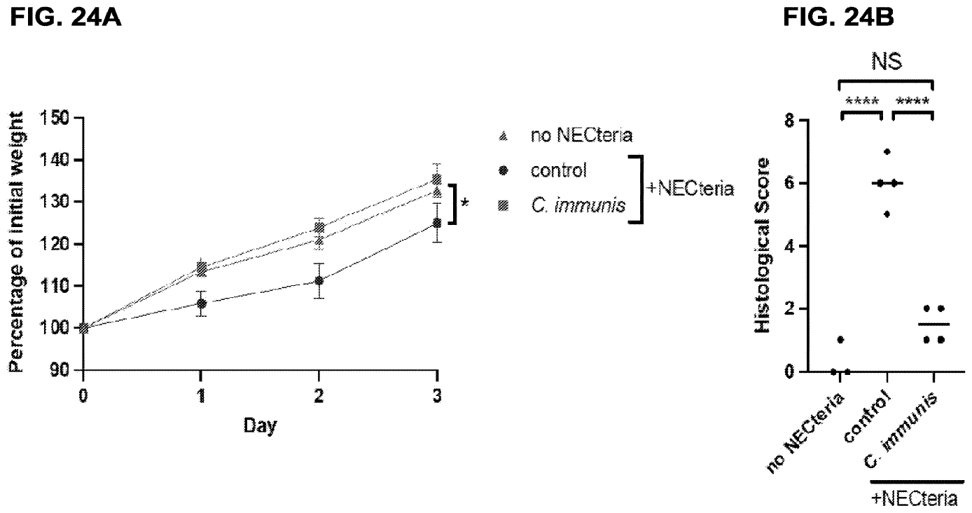

Referring now to FIG. 24, Rag+/− pups (~7 days old) were treated with hypoxia and intestinal pathogens (NECteria) as per Gopalakrishna, Nature Medicine 2019 (DOI 10.1038/s41591-019-0480-9) to induce necrotizing enterocolitis. In FIG. 24A, mice were treated with either a control or C. immunis as a Probiotic and body weight was monitored over 3 days. The C. immunis treated pups had body weight gain similar to control animals (that did not receive the NECteria organisms) and, as shown in FIG. 24B, lower inflammation on intestinal histology compared to the control animals.

C. immunis Protects Against a Second Model of Necrotizing Enterocolitis

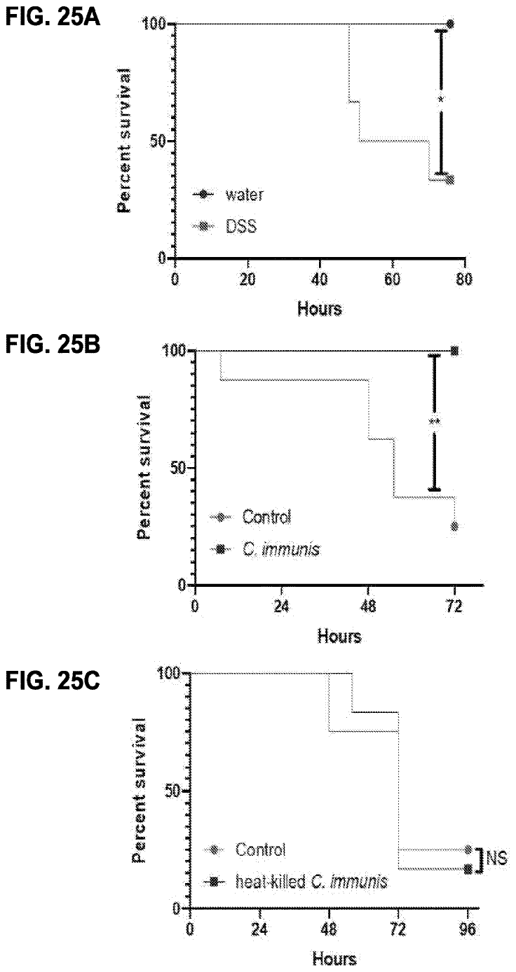

Referring now to FIG. 25, neonatal mice were administered dextran sodium sulfate (DSS) by oral gavage, which causes them to die (FIG. 25A), consistent with earlier studies demonstrating that DSS results in a NEC-like phenotype in neonatal pups. Pups treated with C. immunis were protected against disease (FIG. 25B), and this protection required the C. immunis to be viable (FIG. 25C). Of note, these data indicate that any polysaccharide that is present on the organism is functionally inactive, otherwise the heat-killed organism should have retained activity.

### E. Example 5—Enteric Infection with Clostridioides difficile

C. immunis Protects Against Enteric Infection with Clostridioides difficile:

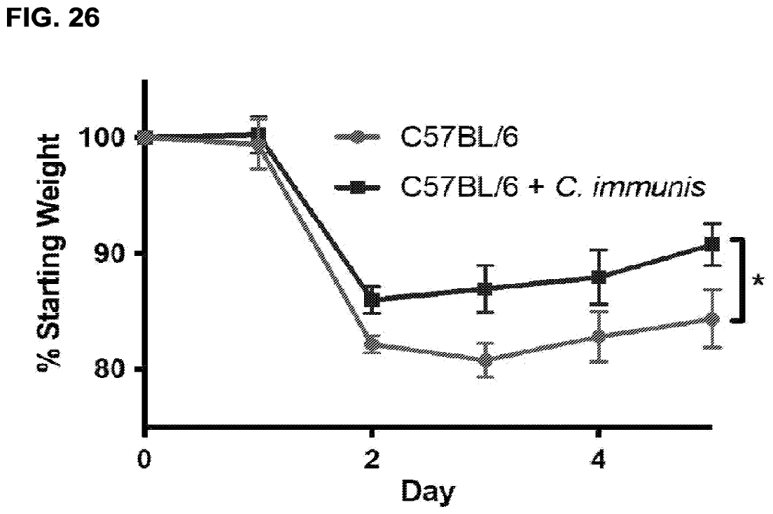

Referring to FIG. 26, mice were treated with or without C. immunis starting 1-week prior to infection. At day 0, all mice were orally infected with C. difficile, and weights were monitored for 5 days. Mice treated with C. immunis had less weight loss than control mice.

### F. Example 6—Structural Data

Glycosyl compositional analysis was performed on the EPS from C. immunis and C. symbiosum to gain insight into the structural determinants required for activity. Interestingly, the overall monosaccharide composition did not differ markedly between the two species, with both EPS structures composed primarily of mannose and glucose (FIG. 15A). The ¹H-NMR spectra for both EPS purifications were very similar with respect to the anomeric signals that form the carbohydrate portion (FIG. 15B-FIG. 15D). However, the C. immunis EPS contained a narrow signal at 3.22 ppm indicative of phosphocholine (FIG. 4A); this sharp singlet was absent in the C. symbiosum EPS, which instead has a triplet slightly downfield (FIG. 4A). Of note, phosphocholine moieties on polysaccharides (e.g., capsule, lipooligosaccharides) from mucosal pathogens enhance virulence by modulating the host immune system, an important precedent for an analogous role that phosphocholine may play in regulating host-commensal interactions.

C. immunis EPS has a Mixed Distribution of Sizes:

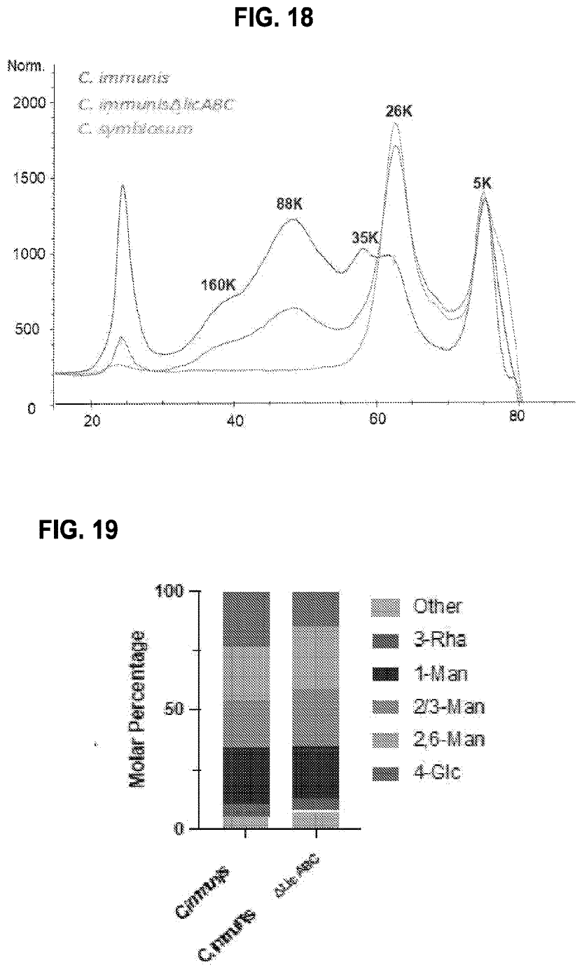

To gain insight into the size of the C. immunis EPS and whether this contributed to its activity, size exclusion chromatography (SEC) of EPS isolated from C. immunis, C. immunis_{ΔlicABC}, and C. Symbiosum was performed. For these analytical experiments, 100 μg of each sample was injected into a Superose 6 column. Elution was performed with 20 mM ammonium acetate as the eluent, with a flow rate of 0.25 mL/min. A series of dextran standards of known average molecular weight were used to estimate molecular weight based on retention time. EPS from the wildtype and mutant C. immunis strains had significant peaks with estimated molecular weights of 160, 88, 26, and 5 kDa, while the C. symbiosum EPS had peaks of 26 and 5 kDa. Intriguingly, the C. immunis EPS also had a unique peak at 35 kDa. Although the C. immunis EPS appears as a high-molecular weight species by SDS-PAGE (FIG. 14), these SEC data demonstrate that it exists as a mixture of different sizes (FIG. 18). Given that the C. symbiosum sample had a lower-than-expected amount of sugar in the sample due to some residual protein and lipid contamination (data not shown), the lower molecular weight peaks at 26 and 5 kDa may represent contaminants rather than the polysaccharide itself. However, the 35 kDa peak observed in the C. immunis EPS and conspicuously absent in the C. immunis_{ΔlicABC} EPS raises the possibility that this peak represents the active fraction.

C. immunis EPS has a Mannan-Like Structure:

To determine the substituent sugars included in the C. immunis EPS and to determine whether these differed from the EPS of C. immunis_{ΔlicABC} or C. symbiosum, we performed glycosyl composition analysis of these 3 polysaccharides. This analysis was performed by combined gas chromatography-mass spectrometry (GC-MS) of the per-O-trimethylsilyl (TMS) derivatives of the monosaccharide methyl glycosides produced from the sample by acidic methanolysis, as previously described (Santander J, et al. (2013) Microbiology (Reading). 159:1471-1486). Although the C. symbiosum EPS sample had a lower percentage of carbohydrates than the other samples (data not shown), the glycosyl composition of all three samples were quite similar, with mannose and glucose being the major components (FIG. 15A). Although mannan polysaccharides are common in plants and fungi (Garcia-Rubio R, et al. (2019) Front Microb. 10:2993; Voiniciuc C, et al. (2019) Proc. Natl. Acad. Sci. U.S.A 116:522-527), they are rare in bacteria. Rhamnose, which is found in many bacterial polysaccharides and has been shown to modulate the immune system, (Henke M T, et al. (2019) Proc. Natl. Acad. Sci. U.S.A 116:12672-12677; Giraud M F, et al. (2000) Curr. Opin. Struct. Biol. 10:687-696; Mistou M Y, et al. (2016) FEMS Microbiol. Rev. 40:464-479; Oyelaran 0, et al. (2009) J Proteome Res. 8:4301-4310) is also present at appreciable amounts. We determined how the monosaccharides were linked to one another by generating partially methylated acetylated alditols of the C. immunis and C. immunis_{ΔlicABC} EPS, as we have previously described (Heiss C, et al. (2009) Carbohydr. Res. 344:915-920). Linkage analysis demonstrated 4 major types of monosaccharides present in a roughly 1:1:1:1 ratio (FIG. 19). Taken together, these data strongly indicate that the backbone of the C. immunis EPS is comprised primarily of mannose, with the other sugars likely being present as side chains. While there are examples of bacteria-derived mannans, they have generally been found in plant pathogens and cold-adapted bacteria but thus far not in the human gut microbiota (Casillo A, et al. (2021) Biomacromolecules. 22:1445-1457; Chatterjee S, et al. (2018) International immunopharmacology. 60:84-95; Corsaro M M, et al. (2001) Carbohydr. Res. 330:271-277; Corsaro M M, et al. (2004) J. Bacteriol. January 86:29-34; Liu S B, et al. (2013) Appl. Environ. Microbiol. 79:224-230). These data indicate that the EPS from these three strains have a similar structure but differ in functionality.

EPS from C. immunis and C. immunis_{ΔlicABC} have Similar Anomeric Regions:

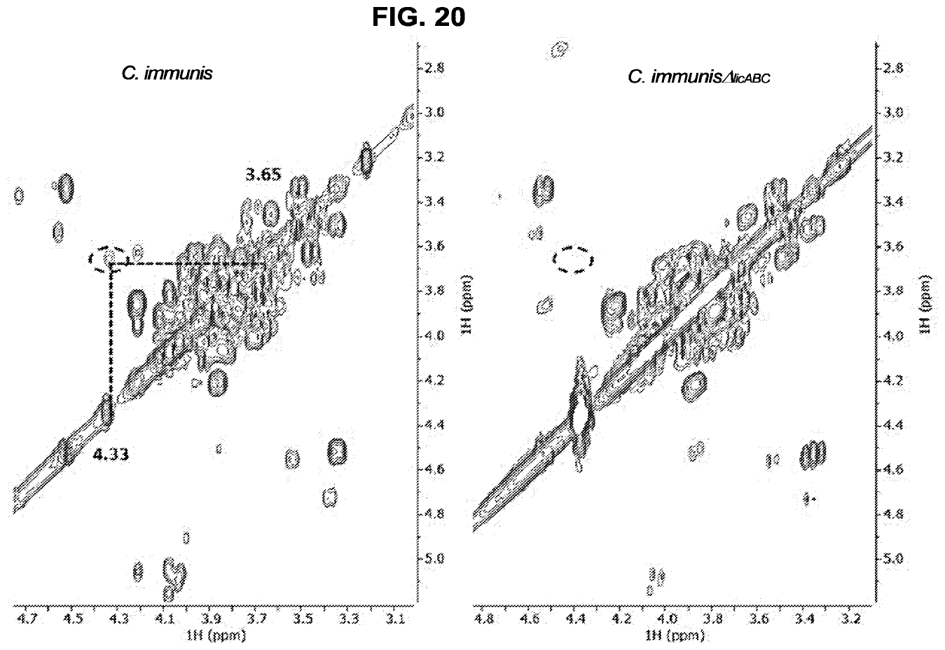

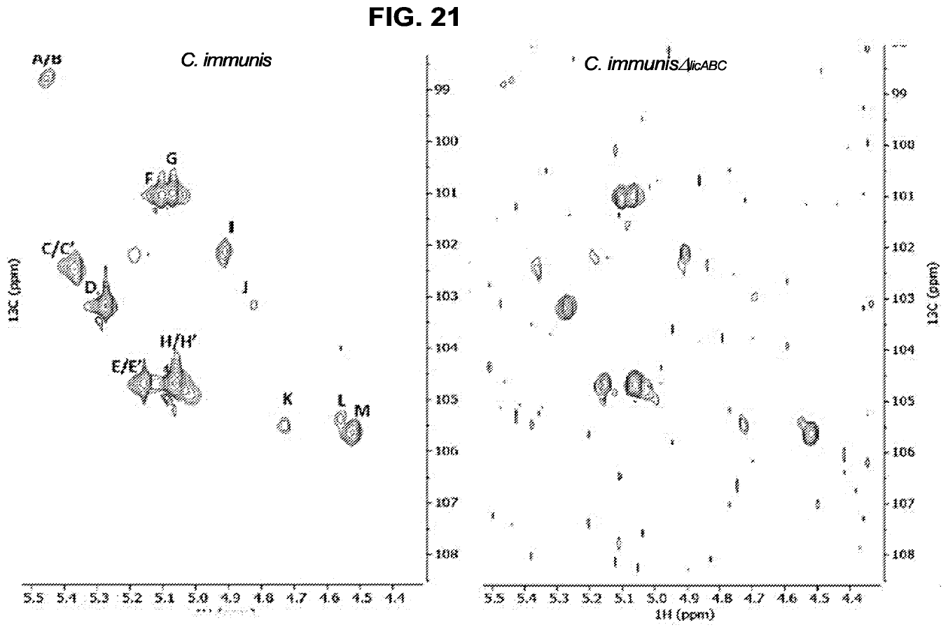

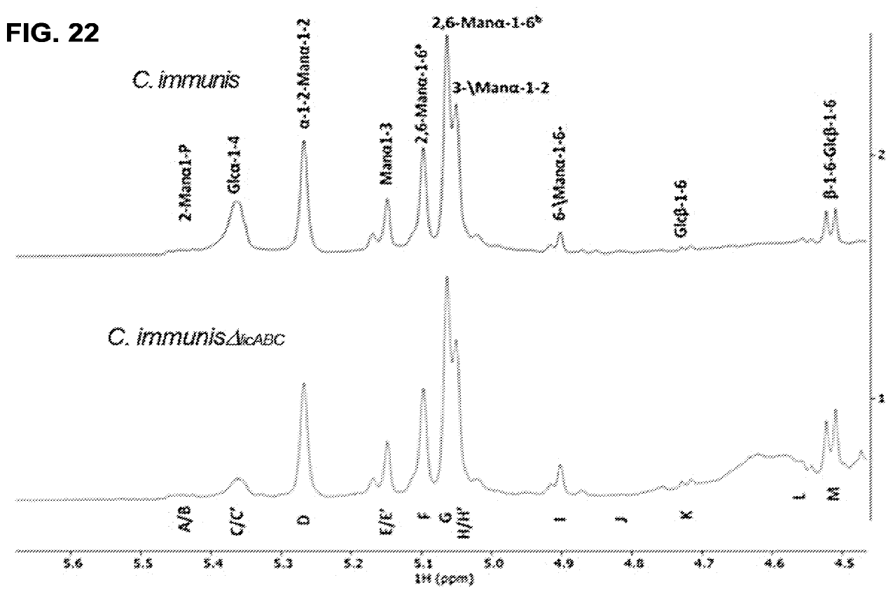

To begin to compare the structures of C. immunis EPS and C. immunis_{ΔlicABC} EPS, we performed correlation spectroscopy (COSY) and heteronuclear single quantum coherence (HSQC) experiments. While COSY reveals correlations between protons that are 2-3 bonds apart (thereby generally allowing assignment of all the protons in a monosaccharide residue), HSQC shows the correlation between a carbon and hydrogen that is bonded directly to it (i.e., via 1 bond). The COSY spectra of the C. immunis EPS demonstrated a cross peak between the phosphocholine protons at 4.33 ppm and 3.65 ppm, which were absent in the C. immunis_{ΔlicABC} EPS (FIG. 20). The anomeric regions of the HSQC spectra indicated no significant differences between the two samples (FIG. 21), though the upfield region again confirmed the presence of phosphocholine only in the C. immunis EPS sample. Together, these data allowed for the tentative assignment of all the anomeric protons in the ¹H-NMR spectra (FIG. 22), which are similar between these two samples.

### G. Example 7—Protocol for Purification of Eps

C. immunis culture was grown overnight (culture becomes turbid with production of gas). The culture was centrifuged to pellet the bacteria. The supernatant was filtered through 0.45 μm and then 0.22 μm filters. The supernatant was concentrated ~15× using a 100 kDa molecular weight cutoff spin column. The concentrated supernatant was treated with DNaseI and RNaseA overnight. The treated supernatant was centrifuged to clarify it. The clarified supernatant was incubated with proteinase K overnight. Ultrapure water was added to the solution to total 15 mL. The proteinase K was removed by spinning the solution through a 100 kDa cutoff spin column. The previous step was repeated two more times to help “dialyze” the solution. The solution was EtOH precipitated overnight. The resulting pellet was weighted to quantify amount of polysaccharide.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A purified exopolysaccharide (EPS) isolated from Clostridium immunis (C. immunis), said EPS comprising a plurality of mannose and glucose subunits and a phosphocholine.

2. The purified EPS according to claim 1, wherein the phosphocholine is present at 3.22 ppm on a ¹H NMR spectra.

3. The purified EPS according to claim 1, wherein the EPS comprises peaks at 160 kDa, 88 kDa, 35 kDa, 26 kDa, and 5 kDa when analyzed for distribution size through size exclusion chromatography (SEC).

4. The purified EPS according to claim 1, wherein the EPS has a mannan-like structure.

5. The purified EPS according to claim 1, wherein the backbone of the EPS predominantly comprises mannose.

6. The purified EPS according to claim 1, wherein correlation spectroscopy (COSY) spectra shows a cross peak between the phosphocholine protons at 4.33 ppm and 3.65 ppm.

7. A pharmaceutical composition comprising a purified EPS as in any of the preceding claims and a pharmaceutically acceptable diluent, excipient and/or carrier.

8. A method of reducing the amount of, activity of, and/or proliferation of colonic group 3 innate lymphoid cells (ILC3s) in a subject, the method comprising: administering to the subject a therapeutically effective amount of the purified EPS as in any one of claims 1 to 6, or the pharmaceutical composition of claim 7, such that the amount of, activity of, and/or proliferation of ILC3 cells are reduced in the subject.

9. The method according to claim 8, further comprising co-administering to the subject one or more additional therapeutic agents.

10. The method according to claim 9, wherein the one or more additional therapeutic agents is administered prior to the purified EPS or pharmaceutical composition thereof.

11. The method according to claim 9, wherein the one or more additional therapeutic agents is administered concurrently with the purified EPS or pharmaceutical composition thereof.

12. The method according to claim 9, wherein the one or more additional therapeutic agents is administered after the purified EPS or pharmaceutical composition thereof.

13. A method of treating or preventing an ILC3-mediated disease in a subject, the method comprising: administering to the subject a therapeutically effective amount of the purified EPS of any one of claims 1 to 6, or the pharmaceutical composition of claim 7, such that the ILC3-mediated disease is treated and/or prevented in the subject.

14. The method according to claim 13, further comprising co-administering to the subject one or more additional therapeutic agents.

15. The method according to claim 14, wherein the one or more additional therapeutic agents is administered prior to the purified EPS or pharmaceutical composition thereof.

16. The method according to claim 14, wherein the one or more additional therapeutic agents is administered concurrently with the purified EPS or pharmaceutical composition thereof.

17. The method according to claim 14, wherein the one or more additional therapeutic agents is administered after the purified EPS or pharmaceutical composition thereof.

18. The method as in any one of claims 13-17, wherein the ILC3-mediated disease is selected from the group consisting of inflammatory bowel disease, psoriasis, asthma, allergy-induced asthma, obesity, multiple sclerosis, enteric infections, necrotizing enterocolitis, and combinations thereof.

19. The method according to claim 18, wherein the ILC3-mediated disease comprises obesity.

20. The method according to claim 18, wherein the ILC3-mediated disease comprises IBD.

21. The method according to claim 20, wherein the IBD is selected from the group consisting of Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease, Behcet's disease and combinations thereof.

22. The method as in any one of claims 8-21, further comprising administering to the subject a prebiotic.

23. The method according to claim 22, wherein the prebiotic is administered prior to the purified EPS or pharmaceutical composition thereof.

24. The method according to claim 22, wherein the prebiotic is administered concurrently with the purified EPS or pharmaceutical composition thereof.

25. The method according to claim 22, wherein the prebiotic is administered after the purified EPS or pharmaceutical composition thereof.

26. A method of treating or preventing metabolic syndrome (MetS) in a subject, the method comprising: administering to the subject a therapeutically effective amount of a purified EPS as in any one of claims 1 to 6, or the pharmaceutical composition of claim 7, such that the MetS is treated and/or prevented in the subject.

27. The method according to claim 26, further comprising co-administering to the subject one or more additional therapeutic agents.

28. The method according to claim 27, wherein the one or more additional therapeutic agents is administered prior to the purified EPS or pharmaceutical composition thereof.

29. The method according to claim 27, wherein the one or more additional therapeutic agents is administered concurrently with the purified EPS or pharmaceutical composition thereof.

30. The method according to claim 27, wherein the one or more additional therapeutic agents is administered after the purified EPS or pharmaceutical composition thereof.

31. The method as in any one of claims 26-30, wherein the MetS comprises conditions selected from the group consisting of increased waist circumference, high blood pressure, elevated serum triglyceride and glucose levels, decreased high-density lipoprotein (HDL) levels, and combinations thereof.

32. A method of decreasing visceral fat in a subject, the method comprising: administering to the subject a therapeutically effective amount of the purified EPS of any one of claims 1 to 6, or the pharmaceutical composition of claim 7, such that the visceral fat is decreased in the subject.

33. The method according to claim 32, further comprising co-administering to the subject one or more additional therapeutic agents.

34. The method according to claim 33, wherein the one or more additional therapeutic agents is administered prior to the purified EPS or pharmaceutical composition thereof.

35. The method according to claim 33, wherein the one or more additional therapeutic agents is administered concurrently with the purified EPS or pharmaceutical composition thereof.

36. The method according to claim 33, wherein the one or more additional therapeutic agents is administered after the purified EPS or pharmaceutical composition thereof.

37. A method of decreasing visceral adiposity in a subject, the method comprising: administering to the subject a therapeutically effective amount of the purified EPS of any one of claims 1 to 6, or the pharmaceutical composition of claim 7, such that the visceral adiposity is decreased in the subject.

38. The method according to claim 37, further comprising co-administering to the subject one or more additional therapeutic agents.

39. The method according to claim 38, wherein the one or more additional therapeutic agents is administered prior to the purified EPS or pharmaceutical composition thereof.

40. The method according to claim 38, wherein the one or more additional therapeutic agents is administered concurrently with the purified EPS or pharmaceutical composition thereof.

41. The method according to claim 38, wherein the one or more additional therapeutic agents is administered after the purified EPS or pharmaceutical composition thereof.

42. A method of inhibiting and/or decreasing the expression of Cd36, Scd1, Nfil3 and/or any target genes thereof, in a subject, the method comprising: administering to the subject a therapeutically effective amount of the purified EPS of any one of claims 1 to 6, or the pharmaceutical composition of claim 7, such that the expression of the Cd36, Scd1, Nfil3 and/or any target genes are decreased and/or inhibited in the subject.

43. A method of inhibiting and/or decreasing the expression of Cd36, Scd1, Nfil3 and/or any target genes thereof, in a cell, the method comprising: contacting the cell with an effective amount of a purified EPS as in any of claims 1 to 6 such that the expression of the Cd36, Scd1, Nfil3 and/or any target genes thereof are decreased and/or inhibited in the cell.

44. A method of increasing the expression of the gene Csf2, and any target genes thereof, in a subject, the method comprising: administering to the subject a therapeutically effective amount of the purified EPS of any one of claims 1 to 6, or the pharmaceutical composition of claim 7, such that the expression of the Csf2 gene and/or any target genes are increased in the subject.

45. A method of increasing the expression of the gene Csf2 and any target genes thereof, in a cell, the method comprising: contacting the cell with an effective amount of the purified EPS of any one of claims 1 to 6 such that the expression of the Csf2 gene and/or any target genes are increased in the cell.

46. A kit, comprising: the purified EPS of any one of claims 1 to 6, or the pharmaceutical composition of claim 7.

47. The kit of claim 46, further comprising one or more components.

48. The kit of claim 47, wherein the one or more components are provided in combinations.

49. The kit of claim 46, further comprising a suitable container.

50. The kit of claim 47, wherein the one or more components comprise reagents.

51. The kit of claim 50, wherein one or more reagents are selected from the group comprising dilution buffers, reconstitution solutions, wash buffers, storage buffers, and control reagents.

52. The kit of claim 47, further comprising instructions for using the components.

53. The kit of claim 52, wherein the instructions are recorded on a suitable recording medium.

54. The kit of claim 53, wherein the instructions are present in the kit as a package insert, in the labeling of the container of the kit or components thereof.

55. The kit of claim 53, wherein the instructions are present as an electronic storage data file present on a suitable computer readable storage medium.

56. The kit of any one of claims 46-55, further comprising one or more therapeutic agents.

57. The kit of claim 56, wherein the one or more therapeutic agents are at least one of a biological active agent, a pharmaceutical active agent, an anti-bacterial agent, an anti-fungal agent, an anti-viral agent, a corticosteroid, an analgesic, or a combination thereof.

58. The kit of any one of claims 46-57, further comprising one or more prebiotics.

59. A purified exopolysaccharide (EPS) from C. immunis isolated by the following steps:
culturing medium inoculated with C. immunis;
centrifuging the cultured medium to pellet the C. immunis;
filtering the supernatant;
treating the filtered supernatant with DNaseI and RNaseA;
centrifuging the DNaseI and RNaseA treated supernatant to clarify;
treating the clarified supernatant with proteinase;
dialyzing the proteinase treated supernatant by adding ultrapure water and spinning through a spin column to eliminate the proteinase; and
precipitating the proteinase-free and dialyzed remaining solution with ethanol.

60. The purified EPS of claim 59, wherein the culturing the inoculated medium comprises culturing for between 2 hrs. and 30 hrs.

61. The purified EPS of claim 60, wherein the culturing the inoculated medium comprises culturing for between 5 hrs. and 20 hrs.

62. The purified EPS of claim 61, wherein the culturing the inoculated medium comprises culturing for between 8 hrs. and 15 hrs.

63. The purified EPS of claim 59, wherein the filtering the supernatant is carried out through a 0.45 μm filter, and then through a 0.22 μm filter.

64. The purified EPS of claim 59, wherein concentrating the supernatant is carried out multiple times using a 100 kDa molecular weight cutoff spin column.

65. The purified EPS of claim 59, wherein the treating the filtered supernatant with DNaseI and RNaseA comprises treating for between 2 hrs. and 30 hrs.

66. The purified EPS of claim 65, wherein the treating the filtered supernatant with DNaseI and RNaseA comprises treating for between 5 hrs. and 20 hrs.

67. The purified EPS of claim 66, wherein the treating the filtered supernatant with DNaseI and RNaseA comprises treating for between 8 hrs. and 15 hrs.

68. The purified EPS of claim 59, wherein the treating the clarified supernatant with proteinase is carried out with proteinase K.

69. The purified EPS of claim 64, wherein treating the clarified supernatant with proteinase comprises treating for between 2 hrs. and 30 hrs.

70. The purified EPS of claim 69, wherein treating the clarified supernatant with proteinase comprises treating for between 5 hrs. and 20 hrs.

71. The purified EPS of claim 70, wherein treating the clarified supernatant with proteinase comprises treating for between 6 hrs. and 18 hrs.

72. The purified EPS of claim 70, wherein treating the clarified supernatant with proteinase comprises treating for between 8 hrs. and 15 hrs.

73. The purified EPS of claim 59, wherein the dialyzing the proteinase treated supernatant by adding ultrapure water and spinning through a spin column to eliminate the proteinase is carried out with a 100 kDa cutoff spin column.

74. The purified EPS of claim 66, wherein the adding ultrapure water and spinning through a spin column is carried out multiple times.

75. The purified EPS of claim 59, wherein precipitating the proteinase-free and dialyzed remaining solution with ethanol comprises precipitating for between 2 hrs. and 30 hrs.

76. The purified EPS of claim 75, wherein precipitating the proteinase-free and dialyzed remaining solution with ethanol comprises precipitating for between 5 hrs. and 20 hrs.

77. The purified EPS of claim 76, wherein precipitating the proteinase-free and dialyzed remaining solution with ethanol comprises precipitating for between 8 hrs. and 15 hrs.

78. The purified EPS of claim 59, further comprising the step of quantifying the yield of purified EPS.

79. A method of treating or preventing cancer in a subject, the method comprising:
administering to the subject a therapeutically effective amount of the purified EPS of any one of claims 1 to 6, or the pharmaceutical composition of claim 7, such that the cancer is treated and/or prevented in the subject.

80. The method according to claim 79, further comprising co-administering to the subject one or more additional therapeutic agents.

81. The method according to claim 80, wherein the one or more additional therapeutic agent is an immunotherapy agent.


SEQUENCE LISTING (30 sequences)

SEQ ID NO: 1 - DNA, 20 bp, synthetic construct
  source 1..20: mol_type = other DNA
accacagtccatgccatcac

SEQ ID NO: 2 - DNA, 20 bp, synthetic construct
  source 1..20: mol_type = other DNA
tccaccaccctgttgctgta

SEQ ID NO: 3 - DNA, 24 bp, synthetic construct
  source 1..24: mol_type = other DNA
ctttcaggactaccagacatccaa

SEQ ID NO: 4 - DNA, 20 bp, synthetic construct
  source 1..20: mol_type = other DNA
gatgcaacttccggctacca

SEQ ID NO: 5 - DNA, 23 bp, synthetic construct
  source 1..23: mol_type = other DNA
tcatattgtgcttgcaaatccaa

SEQ ID NO: 6 - DNA, 24 bp, synthetic construct
  source 1..24: mol_type = other DNA
tgtagatcggctttaccaaagatg

SEQ ID NO: 7 - DNA, 21 bp, synthetic construct
  source 1..21: mol_type = other DNA
cttcttctctcacgtgggttg

SEQ ID NO: 8 - DNA, 20 bp, synthetic construct
  source 1..20: mol_type = other DNA
cgggcttgtagtacctcctc

SEQ ID NO: 9 - DNA, 20 bp, synthetic construct
  source 1..20: mol_type = other DNA
cgggcttgtagtacctcctc

SEQ ID NO: 10 - DNA, 22 bp, synthetic construct
  source 1..22: mol_type = other DNA
actggctgttgtccttgagtcc

SEQ ID NO: 11 - DNA, 22 bp, synthetic construct
  source 1..22: mol_type = other DNA
tggaccttccaggatgaggaca

SEQ ID NO: 12 - DNA, 22 bp, synthetic construct
  source 1..22: mol_type = other DNA
gttcatctcggagcctgtagtg

SEQ ID NO: 13 - DNA, 22 bp, synthetic construct
  source 1..22: mol_type = other DNA
gcttgaggtgtccaacttccag

SEQ ID NO: 14 - DNA, 22 bp, synthetic construct
  source 1..22: mol_type = other DNA
actcctcggaacagtttctccc

SEQ ID NO: 15 - DNA, 22 bp, synthetic construct
  source 1..22: mol_type = other DNA
aacctcctggatgacatgcctg

SEQ ID NO: 16 - DNA, 22 bp, synthetic construct
  source 1..22: mol_type = other DNA
aaattgccccgtagaccctgct

SEQ ID NO: 17 - DNA, 22 bp, synthetic construct
  source 1..22: mol_type = other DNA
atccctcaaagctcagcgtgtc

SEQ ID NO: 18 - DNA, 22 bp, synthetic construct
  source 1..22: mol_type = other DNA
gggtcttcattgcggtggagag

SEQ ID NO: 19 - DNA, 39 bp, synthetic construct
  source 1..39: mol_type = other DNA
gctcggtacccggggatcctaataccgtcagctacactg

SEQ ID NO: 20 - DNA, 32 bp, synthetic construct
  source 1..32: mol_type = other DNA
cttcggccggtaggccggacatattctatatc

SEQ ID NO: 21 - DNA, 35 bp, synthetic construct
  source 1..35: mol_type = other DNA
gaatgtgttttttctatatggtgatcttagtaaac

SEQ ID NO: 22 - DNA, 39 bp, synthetic construct
  source 1..39: mol_type = other DNA
agcttgcatgtctgcaggcctttaattggacaaagcgtc

SEQ ID NO: 23 - DNA, 30 bp, synthetic construct
  source 1..30: mol_type = other DNA
gtccggcctaccggccgaagcaaacttaag

SEQ ID NO: 24 - DNA, 34 bp, synthetic construct
  source 1..34: mol_type = other DNA
catatagaaaaaacacattccctttagtaacgtg

SEQ ID NO: 25 - DNA, 333 bp, Bacillus subtilis
  source 1..333: mol_type = genomic DNA
gtgaatgaggaaatggaacaaatcatttttcaaatcatccttcacggaggaaacggtaggagctccgcaatggaggccattgccgctgcgaaaagcggtgacgcagaagaagcgcggaaaaagcttcaagacgctgctgaagaattgtcaaaagcccatcattatcagacagagctgatccaaaatgaagcgggcggagaaaagacggaaatgacgcttctgatggttcatgcacaggatcatttaatgaatgcaatgacagtgaaggatatggctgcagagatcattgagttatatgaaaaaatcaccgagcaaaggggagcaagcatatga

SEQ ID NO: 26 - protein, 110 aa, Bacillus subtilis
MNEEMEQIIFQIILHGGNGRSSAMEAIAAAKSGDAEEARKKLQDAAEELSKAHHYQTELIQNEAGGEKTEMTLLMVHAQDHLMNAMTVKDMAAEIIELYEKITEQRGASI

SEQ ID NO: 27 - DNA, 309 bp, Bacillus subtilis
  source 1..309: mol_type = genomic DNA
atgaatatattactcgtttgtgcagcaggcatgtctactagcttactggtgagcaagatggaaaaaagtgcgcaggaacagggaaaagactataccatttgggcagtttcaggcgattctgtacaaaatcatatcgataaagcggatgtattgcttctcggtccgcaagtccgttacatgcttccgcagctcaaaaaattaggagagtcaaagggggttccggtagacgtcattaataccgtccattatggaacgtgcaatggggcggaagtgctgaaatcagctgaacagcttggacatgtgtcatag

SEQ ID NO: 28 - protein, 102 aa, Bacillus subtilis
MNILLVCAAGMSTSLLVSKMEKSAQEQGKDYTIWAVSGDSVQNHIDKADVLLLGPQVRYMLPQLKKLGESKGVPVDVINTVHYGTCNGAEVLKSAEQLGHVS

SEQ ID NO: 29 - DNA, 1359 bp, Bacillus subtilis
  source 1..1359: mol_type = genomic DNA
gtgaataaagtcaatcagatcttagaagaaaaggtcatgcctatagcgggaagaattgcgggacagcgtcatttgcaggcgctgcgtgatggtatcatcctcacgatgcccctgattatcatcggttcctttttcctcatcattggcaacctcccgatcccgggatacgctgagtttatggcaaaaacgtttggcagctcctggtctgaaaaactggcctacccagtcgacgcgacctttgaaattatgggtcttgtcgccgcgttcggcattgcctaccggctcgccgagaaatacggtgttgatgccttgtcggccggagccatttcccttgcggcatttttattagcaacaccttatcaagtgccgtttatgccggatggcgcgacgaaagaaatcatggttggcggcgggattccgctttcgctcatgggaagcaaaggcttatttgtcgccatgattatcgcaatggtgtccacagagatttaccgccttattatccagcgcaatttagtcttcaaaatgccggacggtgtcccgccggcggtaagcaagtcatttgtcgcgttaatccccggctttgccgtcatcttcctaatttgggcagcccggctgattgttgaagcgacaccgtttgaaagccttcataatattgtaagcgttttattggggactccgctttccattctgggaggaagccttggaggaagccttgtcgccgaagccgtcaaaatgcttttatgggcctgcggccttcacggtgcaaacattgttggcggcgtcatggcaccgatttggtacggcgcaatggatgccaaccggatcgcattccaagccggtgaggagcttcctaagattttcacccagcaatttttcgatatctgggtcaatattggcggaagcggcgccacattggcactggttgtcacgatgttcctgcgcgcccgaagcaaacaaatgaagcagctcgggaagcttgcggtcggcccggccatcttcaatattaacgaaccgattatattcggaatgccgatcgtcatgaacccaatgctcttacttccgtttatcatcacgccgcttgtcacagtgacactcacgtacatcggaatgagcaccggccttgtcgccaaaccggcgggtatcgcagtgccgtggaccatgccgccgatcttttcaggctacttggcgacaggaggaaaagtctcaggcgcggtcatgcaagccattaatattgccgtgtcctttgttgtctactatccattcttcagaatgtgggataagcagaagctgaaagaagaaaacgatctggagctggtccaaacgcctgcagcaacagatgacaaagaagcggctctgtag

SEQ ID NO: 30 - protein, 452 aa, Bacillus subtilis
MNKVNQILEEKVMPIAGRIAGQRHLQALRDGIILTMPLIIIGSFFLIIGNLPIPGYAEFMAKTFGSSWSEKLAYPVDATFEIMGLVAAFGIAYRLAEKYGVDALSAGAISLAAFLLATPYQVPFMPDGATKEIMVGGGIPLSLMGSKGLFVAMIIAMVSTEIYRLIIQRNLVFKMPDGVPPAVSKSFVALIPGFAVIFLIWAARLIVEATPFESLHNIVSVLLGTPLSILGGSLGGSLVAEAVKMLLWACGLHGANIVGGVMAPIWYGAMDANRIAFQAGEELPKIFTQQFFDIWVNIGGSGATLALVVTMFLRARSKQMKQLGKLAVGPAIFNINEPIIFGMPIVMNPMLLLPFIITPLVTVTLTYIGMSTGLVAKPAGIAVPWTMPPIFSGYLATGGKVSGAVMQAINIAVSFVVYYPFFRMWDKQKLKEENDLELVQTPAATDDKEAAL